(19) United States
(12) Patent Application Publication
(10) Pub. No.: US 2005/0260181 A1
(43) Pub. Date: Nov. 24, 2005

(54) Compositions and methods for tissue repair

(75) Inventor: Leonard S. Girsh, Naples, FL (US)

Correspondence Address: IP GROUP OF DLA PIPER RUDNICK GRAY CARY US LLP, 1650 MARKET ST, SUITE 4900, PHILADELPHIA, PA, 19103, US

(73) Assignee: Immunopath Profile, Inc.

(21) Appl. No.: 11/073,514

(22) Filed: Mar. 7, 2005

Related U.S. Application Data
(60) Provisional application No. 60/550,797, filed on Mar. 5, 2004.

(51) Int. Cl. A61K 45/00; A61K 38/46; A61K 31/198

(52) U.S. Cl. 424/93.45; 514/561; 424/94.6

(57) ABSTRACT

This invention relates to a composition and method for treating diseased or damaged tissues. The composition provides anabolic components and other components that mimic conditions of healthy human tissue, promote tissue regeneration and alleviate disease states. The composition is a medicament having a first component comprising a plurality of L amino acids, a second component comprising at least one extracellular matrix compound, a third component comprising at least one polar surface active lipid, a fourth component comprising vitamins, minerals, and trace elements, and a fifth component comprising a probiotic composition.

### CROSS-REFERENCE TO RELATED APPLICATION

This Application claims benefit of U.S. Provisional Application No. 60/550,797, filed Mar. 5, 2004, which is hereby incorporated by reference in its entirety.

### FIELD OF THE INVENTION

The invention relates to compositions and methods which promote tissue repair, which are useful in the treatment of human pathologies, including inflammatory bowel diseases such as Crohn's disease, ulcerative colitis, diverticulitis and irritable bowel syndrome, and in the treatment of autoimmune disease and as in anti-rejection therapy for organ transplant patient.

### BACKGROUND

The treatment of non-infectious disease centers around the position to “kill” as if we are trying to kill an infectious agent. This is exemplified by the discovery that platinum kills bacteria. Many of the leading cancer products are derivatives of platinum (or similar toxic products) such as cis-platinum and carbo platinum.

The prior art teaches a passive relationship, between the genetic code and amino acid structures. However, the prior art does not teach the use of therapeutic compositions for actively enhancing and normalizing functional aspects of the cell nucleus and cytoplasm in disease to stimulate, facilitate, and accelerate protein synthesis in diseased organs and tissues.

Therapeutic stimulant, activator and substrate compositions that provide therapy for hereditary diseases and conditions of hereditary pre-disposition are disclosed in provisional Patent Application Ser. No. 60/149,338, filed Aug. 17, 1999 and are described in co-pending, U.S. patent application Ser. No. 10/269,613, filed Oct. 11, 2002 (each of which is hereby incorporated by reference in their entireties). As there disclosed, molecular monomers having alpha amino- and alpha carboxylic groupings similar in molar ratio of the component amino acid monomers in human tissue and in accordance with the specific code of the 20 amino acids of human tissue can be used for the treatment of diseases.

### SUMMARY OF THE INVENTION

This invention relates to a composition and method for treating diseased or damaged tissues. The composition provides anabolic components and other components that mimic conditions of healthy human tissue, promote tissue regeneration and alleviate disease states. The composition is a medicament having a first component comprising a plurality of L amino acids, a second component comprising at least one extracellular matrix compound, a third component comprising at least one polar surface active lipid, a fourth component comprising vitamins, minerals, and trace elements, and a fifth component comprising a probiotic composition.

The invention also provides a method involving administering the composition to a patient having a damaged tissue. Once administered, the components synergistically interact with one another in vivo to promote tissue regeneration and alleviate disease states or prevent rejection of grafted tissue.

### DETAILED DESCRIPTION

It will be appreciated that the following description is intended to refer to specific embodiments of the invention selected for illustration and is not intended to define or limit the invention, other than in the appended claims.

The invention relates to a unifying medicament composition of matter that serves the basis for synergistic tissue healing tissue regeneration activity analog to and mimicking not only the embryonic stem cell environment, but adding concentrated adaptive components to provide further therapeutic synergy when used alone or in combination with stem cell therapy. The composition is representative of nanobiotechnology, biorobotics, biophysical formulations, in the pharmacodynamics of synthetic stem cell-like medication which is effectual in vivo interactive water bonding and entrapment.

The therapeutic pharmacologic rationale of this therapeutic synthetic stem cell-like 5 component medication: cell bonding forces are dependent upon the five component medication, which are also analog to industrial robotics not only in function and structure but also in function and structure of human and mammalian cytoskeleton such as organelles, nucleus and cytoplasm, and tissue to which they are therapeutically targeted or derived from directly or as a synthetic equivalent templated copy.

Therapeutically this subject composition medication is focused upon healing injured and diseased (including transplated) tissue in contrast to normal tissue. The present composition is characterized as the emulsion and colloidal suspension domain where biophysics, biochemistry, clinical medicine and medical biology and technology meet tissue as tissue gel turgor-bonded and entrapped water forces of the body. This emulsion and colloidal-entrapped and bonded water provided by this subject medication pre- and post-treatment include: (human tissue is approximately 75% H20) can best be measured clinically by studies that “track” magnetic moment of atomic nuclei by nuclear magnetic resonance imaging applying an external magnetic field to an emulsion or colloidal suspension or solution of tissue in a constant radio field and are dependent upon the spin of the hydrogen atom (H+) ion of water in healthy vs. disease environments and upon the turgor-feel of tissue which is lost in clinical dehydration where skin tissue tents when gently pulled. Comparative ultrasound harmonics, which is very sensitive to fluids and tissue texture characteristics, x-ray density may be also measured by x-ray or CAT scan, or comparative carbohydrate metabolism may also be measured by PET scan pre and post subject composition medication treatment.

Pre- and post-treatment studies also include rheologic principles of flow studies which further include: Viscosity measurements, e.g. Brookfield, surface tension measurements, e.g., tensiometer Du Nuoys, and measurement of colloidal and/or emulsion particle repulsion charge of tissue as measured directly by zetameter or indirectly by erythrocyte sedimentation rate and/or zeta crit packed red blood cells divided by the hematocrit controlled cycle centrifuge, and dependent upon Stokes law of settling particles of colloidal suspensions or emulsions, also zeta potential charge dependent as well as gravimetric dependent Svedberg ultracentrifuge sedimentation Sv coefficient of macromolecules. Measurements of comparative tensile strength of healed tissue such as Gardner Mobilometer weighted syringe like plunger device to measure force required to initiate flow.

Further, while not wishing to be bound to any theory, the foregoing appears to be analogous to the progressive formation of stem cells from the human ovum, with replacement of the nucleus with the patient intact nuclear material derived from the patient.

Normal tissue and its components have been shown to offer remarkable disease and wound defense and biofilm corrosive defense comparable but superior to the biofilm defense of stainless steel with alloy crystal lattice structure, analogous to a solid colloid. Liquid crystal of components #1, #2 and #3 are structured and organized like water of emulsion and colloidal structured clathrate water, and differ in entropy from liquid water.

Each biochemical component of tissue and its analog subject composition medication can be shown to play a significant role in tissue healing equivalent to embryonic tissue development.

The subject composition medication mimics normal tissue and synergistically functions in vivo with damages tissue to promote tissue repair.

While not wishing to be held to any theory, this also represents an emulsion and colloidal water bonding domain that functions robotically according to these biochemical tissue component emulsion and colloidal robotic formulations along with surfactant packing parameter zeta potential electro-chemical charges characteristic of life. It can be seen further that the compositions of the invention are structurally and functionally equivalent to the ovum with its nucleus removed in vitro only to be replaced in vitro with the patient's nucleus.

Further, when used in vivo, the vital organelles, DNA, RNA, and ribosomes of diseased and wounded tissue are reincorporated in vivo as revitalized components of this emulsion and colloid domain of life and of normal stem cell tissue.

Component #1, the 20 alpha amino acids, in the non-D form where appropirate, in this therapeutic composition are present in synthetic stem cell-like prescribed molar ratio, specified by the human genetic code of therapeutically targeted tissue and robotics of ribosomes DNA and RNA. Ultimately, these amino acids carried one by one by transfer RNA (which also bind to ribosomes) as amino acyl transfer RNA are transferred with their alpha carbon tetrahedral 3-D fit to the growing peptide chain in progressive protein robotic synthesis utilizing here the amino acids in molar ratio specified by the genetic code of human tissue and the transfer RNA amino acyl transfer is then released from its ribosomal bond. The robotics of these amino acids and the robotics of their protein synthesis due to the administered dosage of amino acid component number 1 working synergistically with the other components impacting upon the law of mass action and equilibrium shift to protein synthesis thereby also inhibiting proteases in countering protein hydrolysis and negative nitrogen balance characteristic of disease and therefore so important in disease management and minimizing side effects of disease associated required medications and important in insertion of protein channels such as osmotic effect including sodium potassium pump and cell membranes (through this osmotic effect and associated zeta potential charge thereby protectively countering pathogenic biofilm and promoting microorganism infective disease resistance pharmacologically characteristic of normal tissue) so completing effects of component number two, and bio-molecular folding resulting bio efficacy and bio function is further dependent upon the comparative lipophilic low HLB<6 and corresponding and surfactant packing density parameter, surfactant number (Ns) equals v/la where v and 1 represent the volume and length of the hydrocarbon CH2 chains C2, C3, C4, C5, C6 (the same amino acid anti- inflammatory basis analog to ibuprofen) of amino acids of component one, the surfactant lipophilicity and a is the area per polar head and relates to the hydrophilic moiety of the amphiphilic molecular structure to aggregate architecture resulting in a surfactant packing factor number>1. This also determines the repulsion between highly charged zeta potential (measurable by zetameter) surfactant coated surfaces potential charges dependent upon degree of hydrophilicity of the surfactant and on the packing density at the surface with reversed hexagonal liquid crystal micelle aggregation as per polarizing microscopy, x-ray diffraction and magnetic resonance geometry and clumps of emulsifier in equilibrium with surplus of water.

In health and its contrasting counterpart disease, bound water and particularly frequency modulation of water, H2O, pivotal in importance in magnetic resonance as well as all diagnostic procedures as well as a pharmacologic bioengineering science in countering disease.

These core amino acids components comprise more than 80 percent essential amino acids (noteworthy essential amino acids exception such as lysine being a highly hydrophilic exception, histidine also being hydrophilic ) contributing to the lipophilic central protein folded core.

The robotics of the biomolecular folded protein contributed to the more than 80 percent non-essential (to more hydrophobic exceptions glycine and cysteine) amino acid biomolecular folded protein periphery whereby robotic clathrate cage of structured organized water molecules (thereby lower random dependent entropy) water molecules form around the hydrophobic protein molecular core which are reciprocally dependent upon the comparative hydrophilic high HLB of more than 13, and a surfactant packing density parameter and surfactant number of less than 0.5 surfactant number (Ns) equals v/la where v and 1 represent the volume and length of the hydrocarbon CH2 chains C2, C3, C4, C5, C6 (the same amino acid anti- inflammatory basis analog to ibuprofen) of amino acid of component one, the surfactant lipophilicity and a is the area per polar head and relates to the hydrophilic moiety of the amphiphilic molecular structure to aggregate architecture resulting in a surfactant packing factor number<0.5 with resultant repulsion forces between highly hydrophilic surfactant zeta potential charged surfaces with hexagonal liquid crystal micelle aggregation as per polarizing microscopy and x-ray diffraction geometry

Cell membrane CM-self vesiculating robotics of component number two such as PC with different biophysical parameters with intermediate but with hydrophilic preponderance of 8 to 11 HLB and a surfactant packing density parameter and surfactant number of 0.5 to 1 surfactant number (Ns) equals v/la where v and 1 represent the volume and length of the hydrocarbon CH2 chains C16,C 18 of component two surfactant lipophilicity and a is the area per polar head and relates to the hydrophilic moiety of the amphiphilic molecular structure to aggregate architecture resulting in a surfactant packing factor number 0.5 to 1 with resultant comparatively more balanced forces between lipophilic and hydrophilic with hydrophilic preponderance surfactant zeta potential, charged surfaces, with bilamellar/bilayer geometric liquid crystalline phase micelle aggregation resulting in robotic self vesiculating cell membrane formation per polarizing microscopy and x-ray diffraction with milky dispersion appearance macroscopically.

These robotic functions offer further disease management opportunities in their high HLB hydrophilic such as lyso-lecithin, Tween 80, sodium lauryl sulfate which may be used in combination to reduce LD 50 or comparatively alone to modulate and normalize mitosis and apoptosis, and in normalizing abnormally folded protein molecules such as but not limited to Alzheimer's disease, Mad Cow disease and its human equivalent.

ECM-collagen, proteoglycans aggregate complex of post-translational nonrandom macro-molecular glycoproteins, and sulfated aminated polysaccharides, constitute therapeutically healing injured and diseased emulsion and colloidal tissue gel turgor bonding of water forces of the body. This emulsion and colloidal bonded water provided by this subject medication (human tissue is approximately 75% H2O) can best be measured clinically by studies that tag magnetic moment of atomic nuclei by nuclear magnetic resonance imaging applying an external magnetic field to an emulsion or colloidal suspension or solution of tissue in a constant radio field and are dependent upon the spin of the H+ion of water in healthy vs. disease environments and upon the turgor feel of tissue which is lost and in dehydration skin tissue tents when gently pulled. Viscosity measurements e.g. Brookfield, surface tension measurements e.g. tensiometer Du Nuoys, and measurement of colloidal and/or emulsion particle repulsion charge of tissue as measured directly by zetameter or indirectly by erythrocyte sedimentation rate and/or zeta crit packed red blood cells divided by the hematocrit controlled cycle centrifuge, and dependent upon Stokes law of settling particles of colloidal suspensions or emulsions, also zeta potential charge dependent as Svedberg sedimentation coefficient of macromolecules.

The glypicans ECM component with its characteristic lipid foot cell membrane anchor amphiphile-stabilized cell and tissue interface emulsion robotic bonding forces under biophysics control of the foregoing surfactant packing parameter and Number(s) which equals v/al further functions as one of these three polar surface active lipid components, are further bonded by the colloidal, extracellular matrix and associated enmeshed growth factors, highly charged polymer particule absorption to the interface which also stabilizes its emulsion cell membrane intimate contactant effect. This extracellular matrix functions analog to an avian nest in its protective bonding of the stem cell and its biologic equivalent fertilized egg. However at this biomolecular level these interactive noncovalent and bonding forces (only ten times less than covalent bonding forces) of hydrogen bonding, electrostatic forces, van der Waal forces provide the three respective component (including amino acids and ECM glypicans) emulsion polar surface active lipid surfactant packing parameter activity acting collectively with colloidal gel ECM in providing the essential bio molecular bound water and its resultant tissue turgor in healing and tissue regeneration and providing the added at least 48% increase in tissue tensile strength in healing and tissue regeneration of diseased and wounded tissue synthetic stem cell-like therapeutic subject composition medication. While not wishing to be bound to any theory, the foregoing appears to be analog to the progressive formation of stem cell analog to the formation of the stem cell from the human ovum with replacement of the nucleus with the patient intact nuclear material derived from the patient.

All the foregoing components are anabolic. This restriction of the use of catabolic products and microorganisms and disease debris to prevent fouling the bio-membranes in an analog fashion to fouling of industrial ultrafiltration membranes as used in the dairy industry.

These bonding forces' bio efficacy keep us from being just a clump of cells and molecules. This can be shown experimentally in the fertilized sponge egg stem cell when the extracellular matrix biochemical collagen, glycoproteins fibronectin, laminin is filtered away from the cells a clump of disunited cells result only to reform these ECM biochemicals reunite (and reform in a few hours) associated with the reuniting adhesive forces of these foregoing extracellular matrix glycoproteins. This effect was also seen in a patient on long-term steroids wound laceration and swelling of the site was treated with warm compresses was repeatedly was shown to wash away these foregoing adhesive glycoproteins such as fibronectin and laminin in that the wound separated. This effect was progressively reversed omitting warm compresses, by use of synthetic stem cell-like therapeutic subject composition locally and systemically along with Steri strips

It is in this same synthetic stem cell subject composition setting whereby component number one, in a dosage of at least 5 to 10 grams to 25 grams non D amino acids present in the specific genetic code molar ratio of human tissue impacts through the law of mass action synergizing and enhancing the efficiency of the natural ribosomal robotics of tissue proteins synthesis. Higher doses such as 90 to 150 grams or even 100 to 200 grams for these components come up four to six times in a twenty-four hour period can also be administered. In fact, higher doses may be required in severe cases to avoid the need to for organ transplantation, such as liver transplant. Onset of protein synthesis is further triggered by phospholipase A2 release from PC of lysolecithin with a high HLB of 15 and (dosage 0.5 to 1 g and as high as 5 to 10 grams) as seen clinically in the GI tract with this subject composition and in the fertilized ovum at the time of sperm penetration. This therapeutic agent also impinges upon and expands the genomic expression of great value therapeutically in the management of diseases with genetic predisposition. These therapeutic compositions after use for as long as one month exhibited this effect clinically even though avoiding these specific medication compositions for as long as six months.

These therapeutic pharmacologic features, focused upon disease deficient or a secondary side effect of the multiplicity of medication such as but not limited to anti-inflammatory drugs such as aspirin or ibuprofen significantly interfering with protein synthesis, associated with negative nitrogen balance. This may be readily countered Cantor by significant protein synthesis effect derived from component number one may additionally be advantageously and simultaneously coupled with its component short-chain C2, C3, C4, C5, C6 organic fatty acid feature of genetic code amino acids of component one. This may be exemplified by the eight C3 propionic acid derivatives such as tyrosine which are analog to the anti-inflammatory activity, structure and function to such anti-inflammatory drugs as ibuprofen. These anti-inflammatory activities have been documented as 80 percent efficacious in reduction of inflammatory chemokines, all also present possessing the above protein synthesis activity (due to the tetrahedral fit of these 20 non D specific to the human genetic code human) and not noted in the prior art with anti-inflammatory drugs when this therapeutic composition is in-vitro incubated with inflammatory tissue such as Crohn's disease tissue.

It is these same components, illustrated in component #1, that have been shown to offer the patient radiation protection to withstand radiation exposure injury with particular regard to the NH2 ionizing reactive moieties and SH-reactive moieties of cysteine and methionine of component #1. Additionally, component #1 provides protective non-covalent bonding of DNA, RNA and ribosomes, as well as initiating amino acid of component #1 DNA repair. This is furthered by DNA protective anti-oxidant composition, seleno-cysteine and seleno-methionine and retinoic acid vitamin A as beta carotene. The cell membrane phospholipids of component #2 in turn protect these SH and amino groups of component #1 from radiation-induced oxidation.

Synergistically, component #1 may be furthered by components #3, ECM (extracellular membrane) and component #2 cell membrane, (CM) such as (phosphatidylcholine) PC. Component #3 ECM for example, has been shown to increase tensile strength of healing tissue by 48% to counter interference of healing by cortico-steroids.

The three component medications of this series of invention embodiments with emulsion technology and colloidal suspension water, H2O, bonding, offers protective microorganism resistance as microcosm nanotechnology. This microorganism resistance as clathrate bonded, organized, structured water, with entropy and function, different than liquid water, as a liquid crystal emulsion technology and colloidal suspension, is remarkably superior in this microorganism corrosive resistance in contrast to its stainless steel alloy solid colloid analog of iron, carbon nickel, chromium, derived from the periodic table. Further, this subject medication composition is in contrast, derived from the biologic vital periodic table (see, e.g., the components considered GRAS as listed in 37 C.F.R. §1).

This treatment is concordant with therapeutic principles in managing patients using meticulous avoidance measures of disease contributing and/or disease producing catabolic agents thereby minimizing the disease load. This includes strict adherence to avoiding errors of omission such as anabolic agents and their patterns, and co-mission of introducing catabolic agents such as microorganisms with their D amino acid lipid A and/or toxic shock LP S lipoprotein polysaccharides, disease debris or with foreign proteins not in accord with the patient's specific genetic code that would bring about the potential of severe rejection anaphylactic allergic reactions or with diluting away with components other than these five active components specific to this stem cell-like pharmacologic medication healing regeneration process.

Applications to this technology include synergistic augmentation of stem cell and stem cell variants such as adult stem cells and the patient's own stem cells. This may also include application technologies such as but not limited to actively growing tissue derived from benign tumors such as the Schwann cell that have been removed and kept active in in vitro cell culture augmented with subject composition. This specialized variant of synergistic augmentation subject composition technology application may be used to activate re-myelination in the stubborn persistent de-myelinating diseases such as spinal cord injury and associated quadriplegic state, multiple sclerosis, and ALS. Other benign tumor tissue culture such as but not limited to a lipoma may be so dedicated therapeutically.

Bringing protein synthesis as a means of anti-disease therapy where the molar ratio of the component protein amino acids not only satisfies normal human tissue but also approaches the formation of fetal human tissue in this stem cell therapeutic goal. This protein synthesis also thereby more readily and synergistically satisfies another equilibrium of protein synthesis, and thereby reversing the negative nitrogen balance equilibrium characteristic of disease. This positive nitrogen balance equilibrium satisfies the law of mass action by offering these pre-formed monomeric components of human tissue protein to synergistically expedite complete tissue protein synthesis resulting in a more feasible drug dosage with more patient compliance. A dosage of 50 to 100 grams in contrast to an 80% to 90% larger dose of 500 grams per day which is no longer considered an acceptable dosage for medication.

This subject composition medication is possible due to the synergistic action of the five components of the invention.

These therapeutic compositions are abundantly supplied and are formulated to contain amino acids in amounts that correspond to molar ratio of amino acids in a damaged organ, tissue, or protein. The amounts of each component can be adjusted to match the nature of the organ or tissue being treated. In reversing disease through this series of inventions, major side effects can be greatly minimized with co-use or sole use with these therapeutic compositions.

It is not only in the applied biochemistry and its associated biomolecular structures but also the biophysical surfactant functions including surfactant packing parameters and particle charge of the first three component compartments and particularly the key to this fluid dynamics fluidizing and hydrophilizing at code of osophical therapy (also present in components one and three with the most concentrated surfactant function in two) can be poignantly modulated even with the challenge of modulating and thereby normalizing the abnormal mitosis of cancer through the biophysical function and structure of the polar surface active lipids in component number two along with maturation factor of ethylene oxide Tween 80.

The amount of surface-active polar lipid to include in the composition can be determined by viscosity measurements. Tissue concentration can be measured by viscosity (as used in blood serum which normally is 1.12 to 1.22 centipoise with upper limit of three). In the case of the intermediate HLB 8 to 11 (as exemplified by PC phosphatidylcholine when so used) circulation is improved 25% however there is no change in viscosity or the red blood cell sedimentation rate at these HLB ranges because of the fact that biophysical functional effects is upon the cell membrane. With its use the red cell membrane becomes more plastic, and is made more pliable thereby enhancing circulation and oxygenation.

Providing a polar surface active lipid liquid crystal surfactant of extreme HLB to overcome the disturbed fluid balance and lack of fluidity of the biophysical inertia of the non metabolizable necrotic debris of the disease process results in a crystal (such as but not limited to calcium phosphate crystal where the phosphorylase enzyme which in turn releases phosphate to produce the insoluble salt deposit of calcium phosphate).

MRI crystalline calcium salts detected by MRI in the coronary artery may make stress testing not necessary. And biochemical models so derived from the crystallization requirements (as historically in the case out of the x-ray diffraction study of the DNA molecule) may lead to the biomolecular engineering model of life but the possibility of the disease variant of life (in contrast to normal model of life) must be given serious contrasting consideration.

Other intracellular and tissue body deposition responses include the lipid cholesterol crystal found in atherosclerosis and coronary artery disease whereby the lipid crystal has a melting point of 50 degrees higher than normal body temperature. Other crystal responses included poorly soluble uric acid crystal deposits derived from purine metabolic products or exogenous derived silica crystal and asbestos bodies and other difficult to process shards resistant to fluidity necessary for normal metabolic processing. These perpetuating foreign substances promote chronic inflammation, chronic granulomatous reactions, and in certain situations (such as but not limited to asbestos) may progress to cancer after a long period of deposition (which may be as long as 20 years). “Debris” may include materials produced from poorly attainable or derivable processing due to lack of metabolic tools (such as a carbohydrate and glycogen trapped as polymerized glucose form of energy not obtainable from glucose because of the lack of insulin receptor response, as in the case of Type II diabetes, or deficiency of enzymes, as in the case of “storage diseases”). In the case of trans fats, it has been observed to be associated with Type II diabetes with poor insulin receptor response even though production of insulin is adequate. It is likely that trans fat deposits, without adaptable trans fat enzymes, and again with 40 to 50 degrees melting point higher than body temperature, may be amenable to disbursement of the fat with low HLB surfactant followed by further fluidizing the fat with the high HLB surfactant.

Protein, when misfolded, loses its biologic function in diseases such as Alzheimer's disease, Huntington's disease, and Mad Cow (Creutzfield Jacob) disease with resulting neuropathologic response of tangles, which also may be seen with lead poisoning and metals such as aluminum and zinc that are under consideration for their involvement with Alzheimer's disease.

This invention surmounts the awesome challenges of disease treatment by restructuring diseased tissue with biochemical and biophysical components of normal tissue, which have the associated features of restructuring, healing and regeneration of organs and tissue to their normal status. This invention mimics human tissue and thereby draws from normal molecular structured biochemicals with required biophysical function and also from the pharmacopoeia from major industrialized countries to produce the present compositions.

This has been so accomplished with results which include a therapeutic stem cell-like composition which, by simulating, accelerating and facilitating stem cell healing, increases the tissue regeneration capacity of the patient's stem cells, thereby reversing diseases of great severity and complication. For example, organ failure can be reversed without resorting to such extreme measures of desperation and gravity, including organ transplant or tissue graft. As result of this unique focus and sourcing the associated risks and objections of dependency upon the use of human tissue and human embryonic tissue is not required.

This inventor has observed that tissue has a self healing effect promoting tissue healing and tissue regeneration. Not only does it maintain good health but also it has been observed that the patient's blood is withdrawn from patients with a leg ulcer and the blood is then applied to the ulcer the blood is shown to have healing qualities. Cartilage placed in a wound also promotes and accelerates wound healing. The anabolic biochemical and biophysical essence and equivalence of tissue has been found in these embodiments to have the same healing and tissue regeneration pharmacologic qualities, when devoid of genetic DNA mismatch and other catabolic factors, including the catabolic effects of microorganism overgrowth that lacks pro-biotic qualities. The healing efficacy of these tissue components gives us further appreciation of the protective action of human tissue over and above (and other than) the immune protective system, or perhaps may be an integral component of the immune system.

The components are most effective when freely available to the metabolic stream, and thereby overcome the disease producing debris of disease and crystal seeding effect which is obstructive and foreign to the metabolic stream. Mismatching is further assured by adherence to tissue equilibrium, particularly applied here as the hydrophilic/lipophilic balance HLB equilibrium. Therapeutically, through polar surface active lipid surfactants, and other components of the present composition, the tissue can maintain the unique required strata of alternation of hydrophilic with hydrophobic components such as lipids.

This strata is analogous to the earth's strata exemplified by the hydrophobic nucleus surrounded by hydrophobic cytoplasm further surrounded by lipophilic cell membrane and the strata are finalized with a hydrophilic extracellular matrix. The same patterned alternate strata can be seen in the biomolecular macromolecules of proteins with the lipophilic central core derive primarily from the essential amino acids surrounded by the hydrophilic periphery of primarily nonessential amino acids further forming and attracting a clathrate cage of structured ordered nonrandom non-liquid water accounting for the alpha helix or beta sheet folding and associated and dependent biologic structure and function.

It has been found in these embodiments that high HLB surfactant treatment alters the allergenicity of cat protein's 3-D structure and pathogenicity.

These same biophysical features provide the opportunity to use highly hydrophilic surfactants with their high surfactant packing parameters to provide, through hydrogen bonding, a clathrate cage of structured water and energy input and change in entropy that enhances refolding of the misfolded proteins and protein derangements and aggregates that are pathophysiologic and pathogenetic basis for diseases such as Alzheimer's disease, Parkinson's disease, Mad Cow disease and its human equivalent transmissible spongiform encephalopathy (Creutzfield Jacob disease). It is this tissue essence and this biochemical and biophysical molecular engineering that has resulted in therapeutic efficacy combined with bio-safety offering therapeutic opportunities that have been otherwise not forthcoming.

This unique drug discovery technology and characteristics of therapeutic synthetic stem cell-like composition is a healing tissue regenerative therapy and has been shown to be effective in averting organ graft in the replacement of disease ravaged tissue, whether inflammatory, acute, chronically inflammatory, degenerative, neoplastic or genetic pathogenesis or etiologic, on the basis of mimicking human and mammalian tissue. This anabolic tissue copy basis is not only a biochemical copy, but also a functional bio-physical model copy of normal tissue function, with meticulous avoidance of catabolic components, derived from a unique biologic periodic table. The subject composition also permits tissue reorganization, with replenishment not only of the tissue, but even of its trace elements, vitamins and minerals. Additionally the diseased organ or tissue secretions (such as human breast milk) also represent a biochemical and biophysical copy for therapeutic normalization of these tissues.

In producing these copies, the fluidity of function has also been copied by mimicking and preparing an analog copy, and therefore normalizing the hydrophilic lipophilic (HLB) equilibrium balance of the tissue, HLB (with intramolecular 2OH/CH2 ratio of these embodiments exemplified by by normalizing Tween 80) surfactant energy input and associated change in entropy along with any defective human and mammalian tissue equilibria.

In so doing, not only the tissues, cells but even the microscopic and sub-microscopic structure and functions of the cell organelles undergo normalization of mitosis and apoptosis ideally characterized for disease treatment, for example, anticancer therapy. The further normalization of mitosis includes the mitotic organizing centers of centrioles, peri-centriolar clouds, spindles, chromosomes and centromeres (kinetochores) of the chromosomes, acting like seeds of crystallization in conjunction with the microtubules and associated protein with tubulin tread milling polymerization. The mitotic associated tubulin protein of the microtubule has a double origin, the centriolar poles and the chromosome.

This nanogram and picogram pursuit of repair is all based on the atomic and molecular level of human tissue function as illustrated by the synergistic action Component Nos. 1, 2, 3, 4, and 5 of the composition of the subject invention.

Without wishing to be bound by any particular theory, the interaction of components in the present composition can be described as a bio-computer signaling system based on the semi-conductivity bio-computer inter-molecular, therefore intercellular and inter-tissue signaling system, of components of No. 1 and No. 2 and No. 3. The functional biophysical overlapping of these three components is the polar surface active lipid surfactant intrinsic to these foregoing components of an emulsifier the expansion of biochemical surface area interaction by surfactant packing parameters and emulsion system, and most importantly thereby a control of fluidity, metabolic fluidity, metabolism electrochemical charge buildup and enhancement and signaling based on common semiconductor bio computer functionality and obviating, correcting, avoiding crossroads of disease. ECM component No. 3 offers the proteoglycans/complex aggregate to support the colloidal system with similar architectural structural support of structured water, viscosity and lubricant effect of the synovial membrane joints and vitreous helping to hold, for example, the respective retina and umbilical blood vessels in place and unobstructed analog to the cell membrane phospholipids of component No. 2, with hyaluronidase serving as a “colloidifier” analog to a high HLB emulsifier to adjust or reduce and “thin” viscosity to enhance flow.

It has been unexpectedly and surprisingly found that Component No. 5 works synergistically with Nos. 1-4 to further enhance normal tissue function and healing. This fluidizing effect converts roadblocks of disease such as crystals of calcium, cholesterol, uric acid, pigment, disease debris and exogenous crystals such as, but not limited to, silica and asbestos, all acting as disease producing microscopic shards or “thorns” sticking in the metabolic throat and sides of the patient's tissue.

The anti-inflammatory effects associated with all three anti-inflammatory bio physiologic activities and accompaning protein synthesis of components one and two (such as the lyso-lecithin protein synthesis stimulus effects of PC of component two) as but not limited to the contrasting tetrahedral alpha amino acid, non-D, amino acids and non-chiral glycine, fits these tissue 20 specific to the genetic code amino acids in, sharp contrast to the aromatic benzene ring derivatives that do not fit of other inflammatory drugs and therefore also interfere with protein synthesis. Medication side effects are less when co-used with subject composition. Enhancement of enzymatic activity associated with surfactant packing parameters and companion increase in vital zeta potential with use of high HLB surfactants.

The foregoing can be exemplified by non-intrusive, bio-safe, non-coalescent compositions comprising component No. 1, anabolic-non-dextrorotary (“non-D” L amino acids, including but not limited to L-amino acids and non-chiral glycine); component No. 2 (one or more cell membrane components formed by self-vesiculating surface-active polar lipids such as phosphatidylcholine (PC) that forms the double layer of the mammalian cell and nuclear membranes), component No. 3 (extracellular matrix material such as collagen, proteoglycans, chondroitin sulfate, or mixtures thereof), component No. 4 (vitamins, minerals and trace elements), and component No. 5 (probiotic compositions).

### Component No. 1

An anabolic medicament is also provided which is involved in tissue healing and tissue regeneration which, and includes a first component that can mimick the molar ratio of the 20 free non D-amino acids specified in the genetic code of human tissue protein.

These anabolic components may be derived from a “biologic periodic table” with available biochemical formulations of tissue polypeptides and tissue polypeptide proteins from which molar ratios may be readily calculated (sources include the Merck index, the Code of Federal Regulations (CFR 21), and public databases that provide the amino acid sequences of known proteins and polypeptides. Alternatively, suitable ratios or amounts of the non-amino acid can be determined by obtaining a sample of the tissue or tissue type to be treated, and reassuring the amino acid components of the tissue protein using standard techniques.

The anabolic amino acids may be in molar ratio of embryonic fetal neonatal human tissue in the monomeric amino acid form of those listed below. This synergistic human tissue molar ratio, through the mechanism of the law of mass action, can stimulate production of stem cell tissue protein along and promote anti-inflammatory activity through amino acids that are analogous to [symbol] 2, 3, 4, 5 and 6 anti-inflammatory medications.

Embodiments of molar ratios of human tissue include fibrinogen, endorphin, breast tissue and its holocrine gland equivalent breast milk, may include muscle protein such as myoglobin which may be calculated as listed in biochemical text references containing in this case 153 L-amino acids and glycine (SEQ ID NO: 1):

(SEQ ID NO: 1)

GLSDGEWQLVLNVWGKVEADIPGHGQEVLIRLFKGHPETLEKFDKEKHLK
SEDEMKASEDKKHGATVLTALGGILKKKGHHEAETKPLAQSHATKHKIPV
KYLEFISECIIQVLQSKHPGDFGADAQGAMNKALELFRKDMASNYKELGF
QG,

from which amino acid molar ratios are readily calculable.

Bringing protein synthesis as a means of anti-disease therapy where the molar ratio of the component protein amino acids not only satisfies normal human tissue but also approaches the formation of fetal human tissue is one goal of the subject invention. This protein synthesis also thereby more readily and synergistically satisfies another equilibrium of protein synthesis, and thereby reverses the negative nitrogen balance equilibrium characteristic of disease. This positive nitrogen balance equilibrium satisfies the law of mass action by offering these pre-formed monomeric components of human tissue protein to synergistically expedite complete tissue protein synthesis resulting in a more feasible drug dosage with more patient compliance. For example, compositions of the subject invention can contain a dosage of 50 to 100 grams of L-amino acids and/or glycine in contrast to an 80% to 90% larger dose of 500 grams per day which is no longer considered an acceptable dosage for medication.

Additionally, monomeric amino acids of the subject composition can be substituted with other monomeric amino acids. For example: tyrosine (P) (with two hydrophilic hydroxyl groups) is a potential substitute for phenylalanine (F) (with only one hydroxyl group). Similar conservative substitutions include: glycine (G) for alanine (A); methionine (M) for isoleucine (I); glycine (G) for valine (V); aspartic acid (D ) for glutamic acid (E); isoleucine (I) for valine (V); serine (S ) for threonine (T), arginine (R) for lysine (K). Of course, the reverse of these amino acid substitutions can also be performed at the discretion of the practitioner.

The compositions of the invention can include, for example, 10 to 25 grams of molar ratio amino acids such as but not limited to Neocate (SHS, Liverpool, U.K.), which contains the same amino acid ratio as human breast milk, the composition of which is shown in Table 1.

TABLE 1
Exemplary molar ratio of L amino acids
Neocate (15 g/100 ml)

| | Synthetic L-Amino |
|---|---|
| Protein Source | Acids |
| Protein Molecular weight | |
| (daltons) | |
| Mean | 150 |
| % <500 | 100 |
| Maximum | 250 |
| Amino acid profile (mg) | |
| L-Ananine | 91.5 |
| L-Arginine | 162 |
| L-Aspartic acid | 151.5 |
| L-Cystine | 60 |
| L-Glutamic acid | 184.5^{b}[nil] |
| Glycine | 142.5 |
| L-Histidine | 93 |
| L-Isoleucine | 142.5 |
| L-Leucine | 244.5 |
| L-Lysine | 166.5 |
| L-Methionine | 39 |
| L-Phenylalanine | 109.5 |
| L-Proline | 174 |
| L-Serine | 106.5 |
| L-Threonine | 120 |
| L-Tryptophan | 48 |
| L-Tyrosine | 109.5 |
| L-Valine | 156 |
| L-Carnitine | 1.5 |
| Taurine | 3 |
| L-Glutamine | 16.5^{b}[201] |

b Since November 1995 a revised formulation of Neocate has been released. The amounts in brackets indicate the amino acid composition in the new formulation. The patients in this study received the pre-1995 formula composition listed in this table. Source: Excerpt from Table 2 of Bines et al., 1998, J. Pediatr. Gastroenterol. and Nutr. 26(2): 123-128

Again, without wishing to be bound by any theory, it is believed that, furthered by the law of mass action coercing the protein assemblage system, L amino acids and glycine non-covalently bond and fit with the dextro-rotary pentose macromolecules of the protein assemblage system's template DNA and RNA those messenger and transfer RNA and ribosomal macromolecules.

### Component No. 2

Component No. 2 provides polar surface active lipids or liquid crystal micelles with biochemical essences of cell membrane (CM). Component No. 2 can include phosphatidyl choline (PC), omega-3 fish oil, and seed oils.

The highest concentrations of PC are present at birth during youth and young adult phases of life and then decreases progressively until old age. Premature infants are particularly prone to atelectasis or lung collapse, respiratory distress syndrome of the newborn and may be contrasted with full term infants that have adequate PC levels. The sudden rise in saturated PC at 34 to 36 weeks of gestation marks the development of fetal lung maturity. The phospholipids produced represent most of the lipid produced the majority of which is lecithin-saturated PC up to 85 percent of the lecithin, 60 percent of the lecithin is dipalmitoyl PC. Other lipids present are phosphatidylglycerol (PG), phosphatidylinositol (PI), phosphatidylethanolamine (PE). Phosphatidylcholine (PC) can be derived from the soybean plant by degumming followed by acetone extraction.

These highly hydrophilic polar surface acting lipid surfactants also may be utilized therapeutically in treating diseases mediated by mis-folded proteins, including Alzheimer's disease, Parkinson's disease, Mad Cow disease and its human transmissible equivalent.

Polar surface active lipids can contribute, produce and maintain the vital colloidal and emulsion systems of the body. Such polar active surface lipids can be used in accordance with the genetic code and stem cell tissue with outstanding features of promoting tissue healing and tissue regeneration.

Phosphatidylcholine (PC) is present and highest concentration of birth and in childhood where diseases are most reversible and progressively lower concentrations associated with advancing age increased predisposition to disease and cancer and associated syndromes of diseases such as atherosclerosis, coronary artery disease and Alzheimer's disease in accordance with the foregoing embodiments. In fact it is only the aging cow four years or older that is prone to Mad Cow Disease. The association of the progressively lower concentrations of PC with age and its importance in the cell membrane protective barrier further emphasizes the importance of this component in this therapeutic medication composition.

In one embodiment, polar surface active lipid, Phosphatidylcholine (PC) 0.9 g, can be administered one to three times daily, (American Lecithin, Oxford, Conn.) or can be made available in component No. 1 such as in Neocate. Suitable sources of Component 2 include phosphatidylserine (PS) 100 mg contained in a 500 mg complex capsule administered 1 to 3 times daily, (Serinaid, Springfield, Utah); anti-inflammatory Omega 3 fatty acids, 1000 mg per 2 capsules, 2 capsules two to three times daily; 100 mg D-alpha tocopherol antioxidant, antirancidity fish oil complex, with active ingredients 180 mg EPA,125 mg DHA and/or seed oil flaxseed oil (250 mg, organically grown replacing 100 mg of DHA). High HLB polar surface active lipid surfactants such as Tween 80 may also be used.

In cancer with the therapeutic use of highly hydrophilic surfactant such as Tween 80 with its hexagonal geometric format microscopically analogous to normal mitosis, may now be used as a part of the present composition to help fluidize and normalize to the normal metaphase and anaphase stages of mitosis to progress to 2 normal daughter cells instead of being arrested or “stuck”, in an analogous fashion as an old phonographic record might be stuck at the mitosis organization center (MOTC), at which site and transitional time a crystallization like seeding in the growth of crystals effect occurs with regard to the polymerization of tubulin and microtubulin with new tubulin molecules added at the growing advancing end of the microtubules whereas others are lost in depolymerization at the opposite microtubulin end (at anaphase depolymerization at this end of the microtubules occurs), until the “player needle” is advanced or normalized as in the case in cancer with high HLB surfactants.

Variants of Tween 80, a highly ethoxylated high HLB hydrophilic surfactant with 20 moles of ethylene oxide, can be ethoxylated further with 20-40 or more moles of ethylene oxide to increase the HLB and used in the present compositions. Obversely, Myrj represents a low HLB surfactant with 8 moles of ethylene oxide moles to 1 mole of fatty acid such as stearic acid. Two carbon ethylene, (and multiplicity of ethylene oxide derived surfactants), can function as a maturation factor, and may be combined with hydrophilic surfactant activity in these ethoxylated surfactants.

This normal progression of mitosis may be further envisioned as clasped hands which progressively separate at metaphase and the fingers of the clasp hands completely separate and endow each daughter cell with the equal quantitative complement of DNA to continue their genetic activity. A maturation factor is also contained in the same Tween 80 molecule in the form of ethylene oxide (20 moles). The hydrophilicity is further increased not only by the 20 oxygen atoms as H2O in the 20 moles of ethylene oxide and six atoms of oxygen in the one mole of sorbitol but also by the central double-bond of one mole of oleic acid interrupting the 17 consecutive CH2 found in the more hydrophobic stearic acid.

All of the surfactants may be used as the equivalent weight volume dosage as the 0.125 percent dosage in these embodiments, or may be used with a therapeutic dosage of 10 to 20 to 50% of the LD 50. For example, Tween 80 (with a dosage of 20 to 50% of the LD 50) LD 50 in the experimental animals (rats and mice) is 7.5 ml per kilogram (identical to highly lipophilic surfactant PGPR) with a Tween 80 or PGPR dosage of 10 to 50% of the LD 50 can be used. In a 70 kilogram patient the starting dosage total daily dosage would be 50 to 100 ml, further divided into three to four dosages daily. It must be noted that the LD 50 is based upon studies in normal animals with normal hydrophilic/lipophilic equilibrium balance HLB. This specialized use is for patients with abnormal HLB requiring significant hydrophilic surfactant dosage. Therefore this latitude expanding the dosage in these patients is therapeutic in contrast to the LD 50 studies of normal HLB animals that did not require HLB modulation. The LD 50 for sodium lauryl sulfate 1288 mg per kilogram in the experimental animal, (rats orally) with or 900 to 1800 mg, further divided into three to four dosages daily Tween 80. In one embodiment, the low HLB polar surface active lipophilic surfactant PGPR (polyglycerol polyricinolate) can be used at about 0.3%, for example, from about 0.01 about 0.05% or about 10% may be used in any of these applications as a thrust mechanism to disperse and mobilize the hydrophobic tissue components fat 4 to 12 hours before use of foregoing of high HLB surfactant.

Antioxidants such as D-alpha tocopherol 400 units, ascorbic acid 500-1000 milligrams spansule, beta-carotene 10,000 units, along with the L amino acid glycine of this therapeutic composition is also suitable, in particular, for anti-cancer therapeutic application of subject composition.

### Component No. 3

Component No. 3 can include any extracellular matrix (ECM) component, such as glypicans, fibronectin, collagens, proteoglycan, glycosaminoglycans, fibrinogen, and fibrin. Fibronectin may also be used in conjunction with other structural glycoproteins such as osteonectin, SPARC secreted proteins rich in cysteine, osteopontin and osteocalcin, in addition to tenascin present in stem cell containing tissue such as periosteum. These compounds may be used in dosages in this therapeutic subject composition of from about one half to about 2 grams with a range of about 1 to about 50 grams preferably of fibronectin laminin structural glycoproteins, in addition to collagen and associated proteoglycan aggregate complexes, when possible, derived from ECM of amphibian animals such as reptiles and crabs (e.g., stone crabs). With the potential advantages of de-differentiation noted in these animals believed to endow these animals with the ability to re-grow an amputated limb or an eye as in the newt. Sourced when the animals are under the stimulus of re-growing an amputated limb or replacing an eye.

In a further analog fashion, the history of the pharmacognosy teaches the effective use of porcine thyroid in hypothyroidism.

Historically, porcine or bovine insulin has been successfully in diabetes. Liver extract has been used successfully for pernicious anemia. All are derived from the Armour meat packing house source.

All the foregoing stimulated the ultimate detection of the active therapeutic principles.

It is also possible to administer higher doses such as 150 to 200 grams. In fact all of these 5 components may be considered for use in such refractory therapeutic resistant conditions as found in patients scheduled for organ transplant. These extreme dosages, not only of ECM but of all 5 components, may be resorted to with the additional aid of the law of mass action attempting to reverse such resistant conditions. It also should be noted here that in addition to four 740 mg. capsules of shark cartilage, four 750 mg. capsules of bovine cartilage may also be added. This has proved to be of value in two patients with resistant tracheo-bronchitis in that the tracheal origin of the bovine cartilage appeared to have a specific therapeutic synergistic effect. Additionally, this synergistic combination was also more effective in one patient that had arthralgia and muscle stiffness associated with tapering of long term steroids to ½ tablet (23 mg.) 3 times weekly.

Component No. 3 can include all the hydrophilic components of extracellular matrix such as the proteoglycan aggregate complex of cartilage containing hyaluronic acid covalently bonded to extracellular matrix protein and further non-covalently bonded to sulfated GAG such as chondroitin sulfate.

Component No. 3 can be given in the form of 740 mg capsules, 4 to 6 capsules 3 times daily. The capsules can comprise a proteoglycan aggregate complex of cartilage, chondroitin sulfate covalently bonded to core proteins, further non-covalently linked to macro molecule of hyaluronic acid and collagen (see for example, Cartilade, BioTherapies, Inc., Fairfield, N.J.). Component No. 3 is advantageiously used along with component No. 2 self-vesiculating phosphatidylcholine with HLB of 10 to 11 and will further protect the cell and tissue.

Further to the use of the extra cellular matrix component No. 3 for the management of cancer the addition of ECM component No. 3 helps to (1) complete the copy of human tissue; (2) it also adds 50% additional healing capacity to a wound or disease; and (3) it is of great value in correcting the healing deficiency of many patients requiring corticosteroid therapy. It is also noteworthy that the ECM bound water colloidal activity resists transmission of microorganism infection.

The extracellular matrix composition can include (1) fibrous structural proteins such as collagen and elastin, (2) adhesive glycoproteins such as laminin and fibronectin, and (3) proteoglycans and hyaluronan consisting of a core protein and polymers of aminated disaccharides which are also sulfated polysaccharides and glycosylated proteins (glycoproteins).

The sulfated polysaccharides include chondroitin sulfate and proteoglycan complexes of cartilage wherein chondroitin sulfate are covalently linked to extended core protein molecules which in turn are non-covalently linked to a hyaluronic acid polysaccharide glycosaminoglycans polymer molecules with the aid of link proteins.

The extracellular matrix material of Component No. 3 can include, in addition to collagen and elastin, cartilage derived from tracheal rings (of bovine or shark origin) and complex aggregates of very large macromolecule straight chain amino polysaccharide hyaluronic acid polymers of glucosamine and glucuronic acid covalently linked to (proteins and core proteins) and non-covalently linked to chondroitin sulfate. This ECM tissue may also be derived from such sources as animal, plant and micro-organisms that result from normal post-translational protein modifications in the natural production of these ECM components.

Without wishing to be bound by any theory, the function of these extracellular matrix compounds include architectural integrity, imbibing of water as a biocolloid, serving as a lubricated surface (as exemplified by the synovial membranes (and rationale for a therapeutic application in regard to arthritis) and maintenance of viscosity analogous to component number two. Hyaluronidase has been looked upon in the body and therapeutically as a fluidizing, viscosity reducing, thinning enzyme with analog effect of high HLB (15 to 20) surfactants (such as Tween 80 and sodium lauryl sulfate).

A 48 percent inhibition of calcium oxalate urinary tract stone formation was observed in a multi-center study of more than 120 patients given glycoaminoglycans sulfated polysaccharide. The remaining patients formed stones that were smaller and more readily removable in regard to crystal cell adhesion. Similar effects with ECM on blood rheology was noted as with extreme of HLB response with reduction of blood viscosity and lipids as well as anti-coagulant effects.

In other multi-center studies more than 100 patients showed significant improvement in wound healing with a 48% increase in tensile strength of healed wound. Similar effects were noted in controlled animal studies.

### Component No. 4

The 4th component in helping to complete and attain mimicking of normal human tissue comprises vitamins, minerals, and trace elements. Utilizing documented deficiencies of vitamins, minerals and trace elements from available studies or performing pilot study guide lines, suitable compounds and amounts for use in this invention can be readily determined. Exemplary deficiencies in Crohn's disease are documented in the Examples below.

Vitamins, minerals and trace elements can be provided in various concentrations. For example, vitamin B12 (100 micrograms), vitamin A (as beta-carotene 10,000 units), vitamin D, vitamin E, D-alpha tocopherol, Selenium 200 micrograms chelated with methionine as sodium selenomethionine (or to sulfur containing cysteine).

Component No. 4 works synergistically with the other components to provide a therapeutic correction of the major complicating multiple metabolic component deficiencies associated with diseases such as Crohn's disease and pediatric Crohn's disease. Such components are particularly beneficial in the management of regional ileitis as seen in Crohn's disease, in that the ileum is normally the sole site of vitamin B12 absorption and in which vitamin B12 levels are less than ten percent of normal. Of statistical significance, joining a less than ten percent of normal vitamin A level (retinol) correction of which locally and systemically corrects healing deficiency in this disease is associated with long-term steroids along with a less than ten percent vitamin D level, vitamin D, E (D-alpha tocopherol) and prothrombin time in contrast to less than 20% of normal levels of red cell folate, copper, less than 30% zinc, serum folate, plasma ascorbate, less than 50% plasma selenium and hemoglobin.

Other trace elements and minerals and vitamins and enzymes, such as less than 90% serum and plasma glutathione peroxidase, ferritin of a total of 15 studied components) can be seen due to the ravages of disease (such as progressive severe gastrointestinal disease such as the chronic granulomatous inflammatory disease, e.g., Crohn's disease, which specifically in its pathogenesis targets the ileum and its associated negative nitrogen balance. Further complications of Crohn's disease include therapeutic side effects such as the side effects of corticosteroids which include growth retardation and interference with pubertal development.

Component No. 4 can include any of the above. It may be looked upon therapeutically as mimicking these normal components and quantitative levels of vitamins and minerals and trace elements of human tissue.

Deficiencies can be corrected as exemplified by components No. 4 and No. 5 to complete the mimicking and analogous structure of normal tissue in the normal replication of human tissue, normalizing its structure and function in order to bring about the arrest of the vicious cycle of diseases and their pathogenic mechanisms.

Vitamin D supplied in this therapeutic stem cell-like composition can drive and sequester heavy metals, such as but not limited to lead, into the bones by their chelating, thereby greatly minimizing their neurologic to toxic effects.

Additionally, vitamin D can optionally be added to the present compositions to therapeutic replacement enzymes are not available, high HLB surfactant such as but not limited to Tween 80 or sodium lauryl sulfate 0.125% to 1% or 10% to 50% of the LD 50 in normal animals with normal HLBs.

### Component No. 5

Component No. 5 is a probiotic that can include enzymes, such as pancreatic enzymes. It has been unexpectedly discovered that, when administered with Components 1-4, component No. 5 produces a synergistic effect that promotes tissue regeneration, alleviates disease state and decreases dependence on steroids in patients suffering from certain inflammatory diseases, such as a reduced reliance on corticosteroid in Crohn's disease patients. Component no. 5 can include Betaine, HCl, Pancrelipase, Pancreatin 6X (N.F.), Pepsin, Dicalcium Phosphate, Amylase, Bile, Bromelain, Papain, Lipase, L-Glutamic Acid, (ProBio Tex.), Stabilized Probiotic Blend (Each dosage, for example, 200,000,000 pro-biotic micro-flora including Lactobacillus acidophilus DDS-1, Bifido-bacterium bifidum, Lactobacillus bulgaricus, Lactobacillus salivarius), and vegetable and fruit concentrates.

A preferred formulation for component No. 5 includes Phytozyme, (Life Plus Int'l, Batesville Ark.), Amylase 50 mg., Bile 45 mg., Bromelain 30 mg., Lipase 25 mg., Pancreatin 6X (NF.) 100 mg., Pancrelipase 110 mg., Papain 30 mg., Pepsin 70 mg., Betaine HCl 100 mg., and Stabilized Probiotic Blend 20 mg tablet.

Deficiencies of pancreatic enzymes are readily apparent in diseases such as Crohn's disease and cystic fibrosis. Such deficiencies can be corrected here with the present compositions to normalize not only human tissue but its secretions. Reversal to normal flora with pro-biotic is thus desirable and, therefore, is used here as a synergistic component of the present compositions.

This detailed therapeutic replication of normal human tissue secretions and enzymes, deficient in such diseases as Crohn's disease and cystic fibrosis, (therefore, exemplifies the synergistic effect of Component Nos. 1, 2, 3, 4, and 5, which can lead to treatment or reversal of disease states. As discussed in Example 3 below, by including therapeutic components Nos. 4 and 5 and secretions of the tissue and the normalization of the micro-organism flora with associated normalization of function of this gastrointestinal Crohn's diseased tissue has made possible for this patient for the first time to further reduce the corticosteroid therapy (Triamcinalone, generic) for the first time in three decades. The side effects this patient has sustained from long-term corticosteroids has been worsening of osteoporosis documented by two successive bone scans two years apart, recurrent bruising and failure to heal, including the possible need for two skin grafts which this subject composition stem cell-like treatment has prevented.

In the case of the gastrointestinal tract in diseases such as Crohn's disease, the addition of component No. 5, optionally with the addition of pancreatic and enzymatic replacement of deficiencies, normalizes the gastrointestinal secretion component and byproduct of human tissue. The addition of pro-biotic microorganism therapy such as Saccharomyces boulardii helps normalize the abnormal microflora that the disease gastrointestinal tract such as Crohn's disease predisposes to thereby even further normalizing abnormal microflora.

A preferred microorganism is freeze dried lactic acid bacteria, which can be obtained as packets of 450 billion yogurt bacteria. Such bacteria provides a non-toxin producing protective flora that displaces the catabolic flora thriving in the chronic inflammatory debris of a chronic bowel disease such as Crohn's disease.

In addition to the clean up of catabolic debris, the probiotic can deliver anabolic enzymes that synergize with component No. 1, which relates to the amino acids specified by the genetic code. In a yogurt culture, lactobacilli such as Lactobacillus bulgaricus are able to hydrolyze protein such as casein. Streptococcus thermophilus may also be included, providing the ability to utilize proteolytic activity to hydrolyze debris protein. These two starter culture bacteria work synergistically in a 1:1 ratio of Lactobacillus bulgaricus and Streptococcus thermophilus. These bacteria working together efficiently hydrolyze proteins and produce a large amount of free amino acids. L-tyrosine, L-phenylalanine, and L-leucine represent approximately 56% of the free amino acids. However, the proportions of free amino acids can be adjusted. For example, by increasing the proportion of Streptococcus thermophilus relative to L. bulgaricus, the proportion of L-proline can be increased to approximately 71% of the free L amino acid content produced by the probiotic bacteria.

Component No. 5 represents an extension of treatment of the synthetic stem cell therapy subject composition in the same patient as Ex. 1 with the addition of component No. 4 (presented in detail in U.S. patent application Ser. No. 09/639,859, hereby incorporated by reference in its entirety) with the therapeutic component No. 5 enzyme and pro-biotic 0.9 g tablets two tablets daily to three times a day preferably before meals of enzyme replacement and pro-biotic microflora normalizing factor. These favorable conditions make it more and more difficult for the diseased tissue, such as but not limited to chronic granulomatous disease, as in Crohn's disease and thereby reversing the vicious cycle of this disease and other diseases such as but not limited to Crohn's disease. This has proved itself clinically in the embodiment example cited here wherein digestive enzyme formulation containing pancreatic enzyme replacement, (as well as bile which has also been incriminated as deficient in Crohn's disease) along with pro-biotic micro-organism resulted in flora normalization. The pro-biotic in this case was Lactobacillus acidophilus, Bifidobacterium bifidum, Lactobacillus bulgaricus, Lactobacillus salivarius.

The invention relates to a dependent unifying medicament composition of matter which serves the basis for synergistic healing tissue regeneration activity mimicking not only embryonic stem cells but adding concentrated adaptive components to provide further therapeutic synergy, when used alone or in combination with stem cell therapy.

Most importantly component steps are analogous to a team or corporate approach to the anabolic reconstructive reversal of the pathogenesis of a complex catabolic destructive disease. Crohn's disease and many other diseases with such analogous pathogenetic destructive mechanisms, associated enzyme and other deficiencies, and medication side effects, can be treated with the subject composition. In one embodiment, all components of synthetic stem cell-like subject composition formulations are contained in the molar ratios of human tissue.

The human tissue normal molar ratios of these foregoing components include non D-amino acids of the 20 amino acids specified in the human genetic code, polar surface active lipids such as, but not limited to, cell membrane components, extracellular matrix components, vitamins, minerals, trace elements are herein defined as being at least 90% of the composition by weight and 10% by weight or less of composition that is not in conformance with the molar ratios by weight of human tissue. Preferably the human tissue molar ratio of composition of these components are at least 95 percent by weight and five percent by weight or less not strictly corresponding to the molar ratio of human tissue, and most preferably the human tissue molar ratio component composition corresponding to over 99% by weight and 1% or less not strictly corresponding to the molar ratio of human tissue.

The components of the subject invention are preferably combined to form compositions, in particular, composition suitable for human or veterinary use. Such compositions can further comprise a physiologically acceptable carrier or excipient. In certain embodiments of the subject invention, a composition comprising: a) at least one glycosaminoglycan, proteoglycan aggregate complex of hyaluronic acid, extracellular matrix, protein and chondroitin, extracellular matrix compound in an amount effective in the damaged tissue as an anti-neo-inflammatory and anti-neo-angiogenetic agent; b) about one to three grams of at least one polar surface active lipid selected from the group consisting of phosphatidic acid, phophatidylethanolamine, lecithin, phosphatidylserine, phosphatidylinositol, 2-lysolecithin, plasmalogen, choline plasmalogen, phostidylglycerol, diphosphatidylglycerol, sphingomyelin, and any combination of 2, 3, 4, 5, 6, 7, 8, 9, and 10 of said polar active surface lipids; c) a plurality of enantiomerically pure D-amino acids and glycine of about 9 to 25 grams; d) a component selected from the group consisting of polyoxyethylene Sorbitan Monooleate (TWEEN 80), Sorbitan monooleate, grape seed extract, grape extract, and combinations thereof; and e) vitamins, minerals or trace elements selected from the group consisting of Vitamin B12, Vitamin E, selenium, zinc, a probiotic including enzymatic enzymes and combinations thereof is provided.

Components No. 1 and No. 3 can be useful for anti-inflammatory or healing. Component No. 1 can be used to aid in protein formation and component No. 2 can be used to replace damaged cell membranes. Component No. 3 increases tensile strength of wound by 48% in more than 100 patients multi-center and double-blind, as well as in controlled animal studies and component No. 2, modified PC lysolecithin triggers onset of protein synthesis working synergistically with component No. 1.

The full therapeutic formulation of Component Nos. 1, 2, 3, 4 of 5 can further protect from radiation damage as in radiation therapy of cancer and/or radiation in regard to bio-terrorism attacks and nuclear plant accidents. Observations regarding amino acid amino groups and SH groups of cysteine should not exceed 1 g per day, however in the case of cancer, larger dosages to be considered such as 1 to 2 grams daily, indicate that the SH group is further protected by other phosphate groups as in phosphatidylcholine of component No. 2, or by the addition of adenosine diphosphate with the effect of promoting differentiation so important in countering the most aggressive anaplastic aspects of cancer. CSF cytostatic factor may also be added synergistically to compositions for this anti-cancer therapy. This may be derived from the cytoplasmic sap of the unfertilized egg and has similar differentiation promotion factors that are anti-cancer. This unfertilized egg CSF cytostatic, cytoplasmic factor may be sourced and derived from any unfertilized ovum including fish eggs, including sourcing as low allergenic risk potential frogs and/or ostrich eggs since derived from a source where exposure and sensitization has not (or only rarely) occurred.

The compositions offer protective effects including but not limited to the chelating protective effect for macromolecules including but not limited to DNA and their protection from toxic chemicals such as heavy metals as well as antioxidant protection from radiation. The optional addition of antioxidants, such as but not limited to vitamin A (in the form of beta-carotene 10,000 units per day) D-alpha tocopherol, 400 units, ideally chelated to 200 micrograms of selenium to the methionine, per day, ascorbic acid preferably in capsule form 500 milligrams to 8 g in divided dosages is also contemplated by the subject invention.

The inter-biochemical radio-protection of these components of synthetic stem cell therapeutic composition is analog to the protection of aminophostine without the very sickening side effects of nausea and vomiting of aminophostine which may be further minimized (as the case in optional co-use with any therapy with major side effects) by this synthetic stem cell therapeutic subject composition when these three components and specific dosages of subject composition are used.

Liquid crystal high HLB surfactants HLB>13 specifically 15-16 to 20 with a high packing parameter of less than ½ and contributing to a high repulsive of charge zeta potential, along with an increase in surface area and thereby synergize enzymatic activity of enzyme in association with a substrate, can be used as an anti-cancer agent also down modulating mitosis. The use of Tween 80 containing 20 moles (or more) of ethylene oxide a maturation factor is particularly useful in the stimulation of apoptosis, a highly useful anti-cancer feature. The anti-cancer therapeutic features may be used alone or in conjunction with components 1, 2 and 3 as well as components one, two, three, four and five.

These subject compositions may be administered orally or parenterally or locally and in special applications as in anti-cancer may even be administered intra-arterially as therapy used in conjunction with routine medications, to reduce side effects and synergize these companion medications and thereby lessen the dose required of routine medications.

The invention can reverse the need for skin graft in wound treatment of a Crohn's patient. Vitamin A, which may be deficient, can be added locally to anabolicly counter collagenase, which is stimulated by long-term corticosteroids. Anabolic zinc in the form of zinc oxide can also be used in the local and systemic anabolic therapy of the synthetic stem cell-like medicament. These components also help establish or maintain mechanisms associated with the successful reduction in the need for long-term corticosteroid use in 85 percent of the 450 patients studied.

Excisional bowel surgery and correction of fistulization is required in 70 percent of pediatric Crohn's disease patients in a period of conventional therapy five year care; data provided by the Ileitis Foundation of America. The subject composition also provided for a marked reduction in the necessity for major abdominal surgery as exemplified by a 60 percent reduction in the need for correction of fistula by surgical care. In the 40 percent remaining that require major abdominal bowel surgery, this therapy offers a further 55 percent reduction in surgical mortality.

Pediatric Crohn's disease is a disease of hereditary predisposition. However it this specific anti-inflammatory treatment is discontinued after one month of therapy (as might occur in the management children considering stomach tube administration in the past), the absence of recurrence is noted to be as long as six months in those that discontinued treatment (70 percent fortunately do not recur in 7 to 12 months of further observation after discontinued treatment). This is suggestive of a genetic therapeutic component associated with this treatment.

The therapeutic application of the subject compositions also provide anti-inflammatory therapeutic responses without the usual associated complication of impairment of tissue protein synthesis and thereby further aggravation of negative nitrogen balance.

Documented studies showed that further correction of these deficiencies added to any therapeutic plan added significantly to the prevention of this disease's significant predisposition for recurrences. Also included were enzymatic therapy and essential omega-3 EPA fatty acid fats with their contribution to this anti-inflammatory therapy as well as the addition of extracellular matrix (ECM), and reversal of impaired healing (associated with pediatric Crohn's disease and long-term steroids).

The addition of these deficiency corrections would further add to the management of this formerly intractable progressive chronic granulomatous pediatric Crohn's disease in the growing child, potentially contributing to the 15% of patients (vs. the 85%) that were not able to reduce corticosteroid therapies.

As evident from the foregoing, treatment rendered in accordance with the invention is beneficial in several ways. Without wishing to be bound by any theory, provision of component Nos. 1 and 2, in combination with the other components described herein, counter two disruptive equilibrium of disease: (1) the negative nitrogen balance and (2) the disrupted hydrophilic lipophilic balance equilibrium. In so doing I have (1) expanded the genomic environment thereby adding therapeutic elements while minimizing genetic pre-disposition estimated to be present in ⅔ of all diseases, and (2) have corrected the gastrointestinal and subsequent tissue environment initiating the disturbance in the HLB balance.

The present composition incorporates the pivotal strategic components that maintain and form these emulsion and colloidal micellar charged particulate matrix states as an anabolic organized structural and functional cell and its cytoplasmic, nuclear and organelle components along with tissue and organ states that are analog and mimic the component factors and forces of the tissue healing regenerating stem cell. This is in sharp contrast to disease and its associated components factors and forces that contribute to disorganized clumps of cells and their organelle and nuclear contents that not only lack these required unifying forces in disease states, but are also catabolic and disruptive to the state of normalcy and health that stem cell therapy contributes.

Further to this therapeutic end, the synthetic stem cell-like subject composition of each of component 1, 2 and 3 serving as emulsion forming, and thereby unifying, liquid crystal micellar polar surface active lipid with components of No. 3 also contributes to the unifying colloidal state analogous to a unitary modus operandi through biomolecular engineering of the bio function and structure of the stem cell. It is this unique strategized fit with specialized variations (countering through these embodiments specific dysfunctional disease groups), that gives this synthetic stem cell-like therapeutic composition the capacity to mimic the naturally occurring stem cell.

Component No. 2 contains self-vesiculating essence of HLB 8 to 11 or 12, ideally 10 to 11, cell membrane forming and repairing liquid crystal phosphatidylcholine (PC), thereby increasing pliability of the red cell, blood vessel and endothelial membrane enhancing circulatory function by 25%. Component No. 2 optionally contains high HLB surfactants with packing parameters that not only enhance biologic function and efficiency of protein enzymes and their substrate but also promotes protein refolding and thereby normalizing biologic function. This helps to normalize the biologic function of disease promoting protein structures of Alzheimer's disease, Parkinson's disease, and Mad Cow disease. The high HLB (13 to 20, preferably HLB of 15 to 20) liquid crystal surfactants will also enhance fluidity thereby countering debris of disease, and respective seeding of crystallization with reversal of existent crystals. This may be documented by normal viscosity (Du Nuoys of 1 to 3 centipoises). The same therapeutic component modality has been successfully used in-vitro to modulate mitosis with added maturation factor molecular component promoting apoptosis thereby normalizing cancer cells, highly unique, without any prior art anticipation that this polar surface active lipid surfactant would have any anti-cancer effect. These same HLB modulating requirements are used therapeutically here in these embodiments to counter clinically associated diseases such as, but not limited to obesity, atherosclerosis, and coronary artery diseases. In all these therapeutic applications of subject composition (in fluidizing with high HLB surfactant(s)) optional pretreatment with (or administration of) low HLB surfactant(s) to disperse the fat phase can be performed to initiate the fluidizing that high HLB surfactant(s) will finalize in 4 to 12 hours.

Additional discoveries include that these selective concurrent components with the foregoing specific exclusion features not only allow but facilitate, accelerate and synergize tissue healing and tissue regeneration. When combined with component No. 3 (collagen-cartilage) these unique synergistic features permit components No. 1 and No. 2, with L-amino acid and glycine in molar ratios that mimic human tissue, to be used effectively at reduced daily dosages of 10% to 20%, (50 to 100 grams) facilitate patient compliance and do not require hospitalization for intravenous or stomach tube administration. This is contrasted with daily dosages of 500 grams of amino acids in elemental feeding, which are formulated as nutritional food replacement feedings and are met with poor compliance that often require hospitalization for intravenous feedings or stomach tube administration.

This therapeutic composition is directed to the protein assemblage synthesis system and additionally includes all the polar surface active lipid surfactants and L-amino acids and non-chiral glycine (the lipophilic of which is primarily comprised of essential amino acids and constitutes the hydrophobic core of proteins). The hydrophilic components, primarily non-essential amino acids surround and form the periphery of the folded protein macromolecule. These polar surface active lipid surfactant forces are responsible for the final folded protein and its biologic activity associated with zeta potential charged clathrate thereby providing the intramolecular bonding, electrostatic bonding and van der Waal forces with associated energy and entropy forces.

Cell nuclear and organelle membranes with HLB of 8-12 are comprised of polar surface active lipid liquid crystal surfactants, thereby utilizing the same intra-molecular inter-molecular foregoing bonding forces and associated energy and entropy forces, further comprising the omega 3 fatty acid fats (lipase activated in-vivo in the intestinal tract only to be further activated in the cellular membranes as a biologic antagonistic of highly inflammable chemokine mediator prostaglandin two) and the high 13-20 HLB surfactants and the fat dispersing low 1 or 2 to 7 HLB surfactants.

Extracellular matrix polar surface active lipids surfactants further comprising glypicans, utilize the same intra-molecular inter-molecular foregoing bonding forces and associated energy and entropy forces with the associated beneficial function and structure to further modulate vital organelle with particular reference to maintaining the normalcy of mitosis and thereby therapeutic anti-cancer function.

All foregoing polar surface active lipids provide the basis of charged and bonding forces and mechanisms with the unique synergistic component of hydrogen bonding in the clathrate cage structure non-liquid water format.

These bonding features maintain life through its colloidal matrix mediated more so by hyaluronic acid a macromolecule central to the proteoglycan aggregate complex cartilage (imbibing large amounts of water forming a viscous hydrous colloidal gel which gives shock absorbing and lubricant effects, particularly in synovial membrane joint cartilage connective tissue ECM, proteoglycan aggregate complex particularly so with macro molecular bio efficacy of hyaluronic acid biomolecular centrality in cartilage of component No. 3 and component No. 2 emulsion oil and water matrix systems with pivotal effect of the liquid crystal surfactants polar surface active lipids and their highly effective surfactant packing parameters increases surface area and zeta potential hydrogen bonding electrostatic forces and van der Waal forces) and thereby prophylactically and therapeutically lead therapeutic “combat” in normalizing the forces of disease that promote the breakdown of the systems representative of disease, liver disease or skin death. Without these components we would be [symbol] only a lump of cells (Robbins, Harvard edition pathology text).

These three components of therapeutic synthetic stem cell-like subject composition polar surface active lipids surfactant component share semiconductor signaling systems with extracellular matrix component (ECM) component No. 3 (comprising collagen, fibronectin, laminin, and integrins, associated growth factors and protein aggregates including vinculin, talin, alpha actinin) and various combinations thereof signaling protein synthesis (associated with component No. 1), a cell growth and differentiation and motility by collectively initiating and integrating intracellular and intranuclear messages and nuclear signals.

In addition, the liquid crystal high HLB component No. 2 prevents and reverses non metabolizable debris of disease seeded crystals of cholesterol crystals, calcium phosphate crystals, uric acid crystals, pigmentation debris exogenous disease causing crystal shards such as silica and asbestos. The relation of inflammation and cancer can be illustrated by the unfortunate pathologic ending to asbestosis of cancer, mesothelioma, with the clinical therapeutic applications herein of this science and therapeutic synthetic stem cell-like subject composition.

The compositions of the invention can simulate many or all stem cell biochemical biophysical features, as evidenced by averting need for an organ transplant while avoiding key stem cell side effects. Some of the advantages enjoyed by the invention are as follows:

Bioethics independent of use of human embryonic tissue, but can build and rebuild thereby enhancing tissue healing, protein synthesis on existing tissue and in-vitro recombinant DNA tissue culture,

Avoiding the risk of transmission of such diseases as AIDS and Hepatitis and even cancer cells (incipient),

Avoiding the risk of rejection reaction and the need for HLA cross matching,

Adding a significant anti-tumor anti-cancer effect,

Sourcing has avoided the risk of allergic reaction by avoiding protein or substances that would cross match the patient's genetic code.

May be used freely with other medication to reduce their significant risk and dosage of medication.

Other advantages of the invention include:

The subject composition also provides a “unique and novel and exciting in that therapeutic product and action in the patient is dependent upon the completion of the final activity and activation steps and in a sense, the final touches of “manufacturing steps of this therapeutic product occurs in vivo in the patient”.

The subject invention completes the therapeutic composition to make non-healing tissue heal and regenerate.

Prior to the subject invention, those skilled in the art were unable to use Periodic Table as utilized in PDR pharmaceutical and chemical plant whose manufacturing action is complete per se and only in vivo processing primarily concerns excretion and prior inaction.

The subject invention uses pre-made (until available synthetically as in the analog case historically and in the case of past drug discovery and pharmacognosy of thyroid and insulin made available from the major meat packing houses) biologic chemical components with the practicality and safety of GRAS components significantly expediting and maximizing the practicality of new product discovery and development making these products readily available for market and patient use; components No. 1 and No. 2 contain essential components and that the body cannot synthesize (e.g. essential amino acids); component No. 2 contains essential lipids including omega 3 fatty acids (which become activated by lipase pancreatic enzyme in the small intestines and alkaline medium and which are inactive until hydrolyzed into fatty acid and glycerin as exemplified by documentation in heart muscle cells in vitro) in normalizing and preventing fatal dysrhythmia); and component No. 3 contains polar surface active ECM lipid glypicans (one of the three major classes of proteoglycans GAG with its lipid foot anchor on the adjacent (primarily lipid) cell membrane).

Stem cell-caused healing and tissue regeneration can be maintained if contact associated with component No. 3 highly hydrophilic extracellular matrix components along with foregoing glypocans in toto as analog and stimulating the activation of the stem cells in other organs,(specifically exemplified by ECM basement membrane component=Heparan Sulfate GAG a sugar polymer highly polar negatively charged surface effect in common with polar surface active lipids permitting the maintenance of activation of the stem cells of the skin in normal spontaneous skin repair and regeneration after injury). This extracellular matrix basement membrane surface effect in maintaining stem cell character includes interaction with collagen, with several ECM macromolecules illustrating the rationale of the extracellular matrix and component No. 3 stem cell-like subject composition therapeutic effect characteristic including the glycoprotein laminin, the highly sulfated glycoprotein entactin as well as heparan sulfate GAG, extracellular matrix components included in other embodiments. These foregoing proteoglycan are noncovalent electrostatic interaction charged bonds between the negatively charged GAG and positively charged extracellular matrix proteins. Cartilage cells or chondrocytes in a similar ECM contact fashion also remain differentiated only as long as they are in contact with collagen.

Additionally, a similar surface stimulus effect was noted in Steri-strip suture less wound approximation and healing (in view of suture intolerance and breakdown because of prolonged steroid use) wound edges contact approximation, repairing the rift in the basal epithelial proliferating cells and associated stem cells and their stem cell basement membrane maintenance contact with ECM collagen, proteoglycan aggregate complex which stimulated skin cell differentiation accelerating healing, the absence of scar formation, and stopped proliferation and migration of epidermal cells in conjunction with local and systemic therapeutic stem cell-like composition.

In further keeping with the synthetic stem cell-like synergistic formulation component collagen and its associated proteoglycans, the therapeutic stem cell-like composition has been found: (1) to maintain the stem cell activity, (2) to be one of the first substances to be formed after cleavage of the fertilized ovum again showing its importance in the stem cell activity), (3) stem cell activity is again seen when collagen-cartilage is added to wound and thereby stimulates healing, and increase the hill disease or wound tensile strength by more than 48 percent, (4) directing the L-amino acid and glycine to stem cell tissue protein formation is synergized by analoging molar ratios of human tissue profiles (focused in continuation of fetal and embryonic analog to breast milk and breast tissue best directed and utilized in and the specialize in these are the developing embryonic fetus and neonate with the largest population of stem cells). These stem cells activated efficiently with significantly more focused on stem cell tissue protein synergistic through these synthetic stem cell therapeutic subject compositions thereby providing lower effective dosage requirement associated with maximal bioefficacy and biosafety and patient compliance for tissue healing and tissue regeneration than in the nutritional form.

All components, and potential further added components for special disease groups, provide for therapeutic application of the subject technology that when co-used maximize efficiency and therefore lessen the required dosage of each component through their synergy (directed to this stem cell-like therapeutic subject composition dedicated to stimulate, facilitate and accelerate in-vivo the patient's stem cells thereby promoting this tissue healing tissue regeneration effect). This in turn, through progressive intermediate steps, finalizes in-vivo the ultimate activated therapeutic pharmacodynamic medication.

The body's further action on components Nos. 1, 2 and 3 with the optional addition of further components as outlined in these embodiments including components No. 4 and No. 5 to products is devoted to excretion and metabolic degradation that precedes excretion of these products.

I have in conclusion further unexpectedly discovered through these series of inventions many medicaments to help reverse groups of diseases through the medium of this stem cell-like therapeutic composition. The further basis of which is extracellular matrix representation as mesodermal and future mesenchymal tissue which has the ability intact to maintain stem cell activity of the skin. For example, if the skin basal epidermal stem cell layer is separated or severed or broken as in a wound from the underlying collagen proteoglycan aggregate complex of basement membrane stem cell surveillance that healing tissue regeneration is arrested. Molecular embryologic studies prove if the mesoderm and its future extracellular matrix are removed from its normal intermediate contact position these ectodermal and endodermal surfaces degenerate.

Variants of these components with further specialized biologic effect can be found in the developing fetus with the largest population of stem cells, and sourcing from various species so provides representative biomolecularly “origin of species” also seen fetus provides these specialized functions and therapeutic opportunities. For example, the allantoic stage of the developing fetus produces readily soluble allantoin as to animals and birds a product of purine metabolism in its urinary tract, whereas the adult excretes highly insoluble uric acid making some adults prone to gout. By deriving enzymes such as uricase, from such animals, the soluble stage of allantoin can be achieved thereby alleviating the metabolic disability of gout. This analog sourcing of enzymes or synthetic models for synthesis offer many other such examples of therapeutic application.

For example, in addition to shark and cow tracheal cartilage, none of the cartilage at this specified level has proceeded to bone formation. For specialized therapeutic indication and application in impending amputations, such as, but not limited to, the use of stone crab, starfish, (echinoderm biologic class) and newt (salamandridea family, an amphibian), extracellular matrix (or cartilage), with multiple enmeshed growth and de-differentiation biologic factors, from an animal that can replace its own amputated limb and is in the activated biologic process of so doing can be utilized. This initially can be administered as a food and then ultimately desiccated and pulverized in accordance with the state of the art of production of biologic extracts. This de-differentiation extracellular matrix mechanism added to ECM component No. 3 (synthetic stem cell-like therapeutic composition comprising components No. 1, 2, 3) a starting dosage of 1 to 2 grams ideally taking compositely and synergistically with the other three components but optionally may be used alone three times a day could prove of significant value in a patient such as a soldier suffering from impending phases of traumatic amputation on the battlefield. A dosage range 1-50 grams, added to ECM of component No. 3, specializing and varying these options according to the challenging needs of the disease in question.

This may be further exemplified by drawing from the functional advantages of cellular membrane CM component No. 2 with the use of polar surface active lipids liquid crystal high HLB surfactant such as but not limited to Tween 80 in a cancerous group of diseases to modulate functionally than the nuclear organelle in mitotic organizing center mitosis and apoptosis to normalize the cancer cell. This subject therapeutic composition of matter opportunity can be further maximized by comparing therapeutic response results with esterifying the ethoxylated grouping with an additional 20, 40 and 60 or more moles of esterified ethylene oxide to achieve the results desired. Again this therapy may be used alone but ideally further synergized as part of a component No. 2 of the entire three component synthetic therapeutic stem cell-like subject composition. Additional synergistic efficacy of the subject compositions in expanding, synergistically, the genome (potentially mutated) can further normalize DNA along with antioxidants vitamins E, C, and A and synergistically broadening this unique composition for use in anti-cancer activity. Other enzymes otherwise normal but deficient such as but not limited to polymerases may be activated, facilitated, stimulated and synergized by the packing parameter efficiency and increase of surface area by the same polar surface active lipids liquid crystal surfactants. The foregoing are representative of medicaments and drug discovery application technology in therapeutic compositions unanticipated in the prior art.

The same foregoing therapeutic, biomolecular pharmacodynamic application technology may be applied to the proteinopathy pathogenesis of Alzheimer's, Parkinson's, Mad Cow disease and associated human transmissible diseases resulting from mis-folded proteins from Alpha Helix random coil, to abnormal beta sheet, and reverse the folding mechanism with the foregoing high HLB surfactants.

Captured in this medicament are all of the vital healing and tissue regeneration forces of the living stem cell, unanticipated in the prior art, not only strategized, in biomolecular engineering as such but also established bio-efficacy as such. The achievement of this goal with such a degree of bio-efficacy and safety was not only unanticipated by the prior art but this degree of excellence of reproducing the vital force and effect of live tissue was not anticipated by the inventor. It is only through this extension of strategized vital tissue that this healing tissue regeneration therapeutic factor with all its components and bioefficacy can be extended for further continued replication into the tissue itself, an unanticipated accomplishment in therapeutic agents to date.

For example, fetal neonate tissue (whose amino acids molar ratios are analogous to and mimic the tissue and its properties of tissue healing, tissue restoration and regeneration) with further unique combined properties of protein synthesis coupled with anti-inflammatory activity as well as a genetic factor not found in anti-inflammatory medicaments of the prior art. Additionally not found in the prior art is the selective choosing (from the biologic period table) of amino acids in medicament dosage form synergistically adapted to the human stem cell function (in contrast to the multiplicity of nutritionally based elemental feedings used as a medical food rather than a medicament).

I have additionally discovered and utilized a unifying cell and tissue composition that is analogous to the structure and function of the stem cell emulsion and colloidal bonding force and matrix that extends itself to the tissues. The composition can be given, not only parenterally, but more importantly, orally, with further benefit of the oral mucosal delivery system. These bonding forces are liquid crystal surfactant micelle polar surface active lipids incorporate the zeta potential, hydrogen bonding and clathrate structured, non-liquid, water cage, thereby, extending and disseminating this tissue structure bonding and unifying force to the patient's tissues.

Most important of all in this therapeutic breakthrough, unanticipated in the prior art, is the anti-cancer activity, independent of all the prior strategy of killing the cancer as if it were an infectious microorganism, but instead adopting a normalizing factor in the treatment of cancer: modulation and normalizing mitosis using highly hydrophilic liquid crystal micelle surfactant fluidizing (with an ethylene maturation factor) and normalizing the cell and tissue with its progression of maturation to apoptosis.

In regard to pathogenic microorganism antibiotic resistance and testing for same in vitro the in toto combination with the subject composition may counter microorganism pathogenic components such as lipid A and LPS lipopolysaccharide.

Omega 3 oils and Vitamin E (100 units), can be added as a synergistic antioxidant and anti-rancid component further with the omega-3 fish and seed oil synergistic to the anti-inflammatory efficacy of the component 1. In vivo activated, further promulgating and synergizing anti-inflammatory activity without disrupting the essential tissue replacement component of protein synthesis, not found or anticipated in all prior art and anti-inflammatory compounds.

Extracellular matrix (ECM)—only animal tissue of these components—non mammalian thereby any DNA would be unique enough so that not recognized as potentially damaged DNA that could bring about unwanted mutations from which catabolic disease producing factors could be antagonistically derived.

In allergic anaphylactic type rejection like reactions in regard to multiple severe food allergies distant biologic components sourced from non-mammalian animals (such as amphibian derived foods have been found to serve as a universal food donor) can be used. Extracellular matrix components additionally can, preferably, be utilized in this therapeutic composition in the encapsulated powered form (capsules) in contrast to a compressed tablet which have clinically been found not to have reduced bioefficacy.

This continuity of proliferating cellular contact with extracellular matrix collagen proteoglycan aggregate complex as exemplified by, but not limited to the basement membrane and the ECM collagen contact supportive maintenance of the active skin stem cell layer, similar observations have been made in the cartilage cells or chondrocytes correlating ECM contact with similar stem cell activities in cartilage tissue. Similar correlation has been noted with regard to cartilage placed in a wound with activation of stem cells accounting for the stimulation of wound healing associated with about a 48 percent increase in tensile strength of healed wound.

The same therapeutic and prophylactic concept can be used in the unfortunate possibility of a bioterrorism attack (mediated by nuclear, microorganism, or biochemical agents). Therapeutic composition of the subject invention can also be used in the treatment of soldiers on the battlefield.

Optional components for the compositions of the subject invention, include, but are not limited to, non-hydrolysate-derived milk substitutes (preferably free of catabolic products and D amino acids such as microorganism, derived sources). When used in patients with clinically suspected milk allergy or bronchial asthma respiratory tract allergy (such as nasal allergy and hay fever (documentation with allergy skin testing is usually nonproductive)), the patients respond to this therapeutic composition, which may in terms of therapeutic rationale and mechanism response, most probably reside in the anti-inflammatory action, immune modulatory effects completely free of side effects such as commonly seen soporific effects of the antihistamines used for allergic rhinitis, or the side effects of antiasthmatic sympathomimetics and corticosteroids. Also as stressed when used in conjunction with these anti-asthmatic, anti-allergic medications side effects are greatly minimized. This is exemplified by the avoidance of common soporific side effects seen with antihistaminics. As with all these therapeutic applications, their co-use with medications lessens the dosage and the associated side effects.

Catabolic products are only minimally present or absent from the compositions, especially chiral amino acids and racemic mixtures containing amino acids in D form, as well as, e.g., cyclosporin oligopeptides and bacterial cellular walls. Minimally present means in an amount that is less than 10% by weight of the total composition, preferably less than 5% and most preferably less than 1%.

The following catabolic components and factors can counter the maintenance of equilibrium such as hydrolipophilic/lipophilic equilibrium balance factors, which maintain the body's emulsion and colloidal states and that are antagonistic to anabolic tissue components and further unwanted synergism contributory to disease mechanisms: stereo three-dimensional misfits including D-amino acids, and disease response products of debris (that extending disease mechanisms by seeding of crystallization and causing crystalline matter that promotes foreign body reactions of disease) and protein or DNA not in accordance with the genetic code and without any protection for protein mis-folding that promotes crystal shard formation and foreign body rejection reactions of disease. Composition components are optionally inclusive of extracellular matrix post translational protein which is not contrary to the genetic code. Catabolic products further to be excluded: Microorganisms intact or killed as in pasteurized products such as milk and dairy products (such organisms can be excluded via ultrafiltration).

More severe complications of allergic and hypersensitivity diseases may include autoimmune disease such as lupus erythematosis and medication reaction induced false lupus. False lupus has responded to these therapeutic compositions including the collagen proteoglycan aggregate cartilage, chondroitin sulfate complex, thereby avoiding the risks of cortico-steroids, commonly required in these patients, particularly in those patients with the complication of pericardial infusion.

The compositions can also optionally incorporate material that includes stem cells or materials derived from after-birth tissue such as placenta and umbilical cord. The compositions can also include materials that correspond in amino acid composition to mother's milk or to other materials encountered during fetal and infantile development.

The compositions of the invention can also mimic mother's milk or embryonal tissue. This embryonal tissue simultaneously mimics healing tissue, associated with such diseases as inflammation and tissue damage such as trauma, at the same time mimicking and being analogous to mammalian and particularly the human stem cell.

Plant hormones, such as but not limited to, ethylene, abscisic acid (ABA), and gibberelic acid (GA3), a gibberelin, zeatin a (cytokine), auxins (indole-3 acetic acid, IAA) involved in chemiosmotic proton gradients, Zeatin (a cytokine) may be offered in the subject compositions for the prevention or reduction of premature births. The plant hormones may be added to highly hydrophilic surfactants in the modulation of mitosis adding to the management of cancer, and may be incorporated in therapeutic stem cell-like subject compositions, all with a high degree of bio-safety. This is also emphasized relating to other embodiments concerning modulation of mitosis.

The compositions can be employed for local and systemic therapies and can be delivered by topical, oral, parenteral or intravenous routes. In the case of cancer, intralesional or even intra-arterially administration may be practiced. A more preferred route is oral administration, preferably by oral mucosal delivery in which the compositions are formulated into a lozenge or gum that is brought into contact with the oral buccal sublingual, or pharyngeal mucosal surface for a few to twenty minutes (or longer) until absorbed. The high HLB mediated oral mucosal delivery system is as efficacious as parenteral administration of such medications and prophylactic agents as vaccines (further documented by laboratory measured response in other embodiments). When an oral route of administration is used, the component concentrations can be lower than in intravenous routes, since the components do not pass through the liver. This oral mucosal delivery system can also be advantageously used with enzymes or hormones administration. The therapeutic compositions are preferably administered at a temperature slightly less than 100 degrees F., more preferably at or about 98.6 degrees F., to further enhance the synergism of, surfactant and enzymatic activity.

The compositions offer protective effect including but not limited to the chelating protective elect of the macromolecules such as, but not limited to, DNA and their protection from toxic chemicals such as heavy metals as well as antioxidant protection from radiation. The exemplary antioxidants for optional addition to the subject compositions include, but are not limited to vitamin A in the form of beta-carotene, 10,000 units per day; D-alpha tocopherol 400 units ideally chelated 200 micrograms of selenium to the methionine per day; and/or ascorbic acid preferably in capsule form 500 milligrams to 8 g (particularly when uric acid levels are elevated and functioning as a natural antioxidant), in divided dosages. The effects of the compositions can be long-lasting, with benefits extending for six months or more after therapy is discontinued.

The pharmacodynamic basis for successful unexpected therapeutic results with the compositions of the invention include (a) hydrogen bonding, (b) anionic charge, (c) electrostatic polar forces, (d) van der Waal forces, and (e) zeta potential associated with the non-covalent interactions with the macromolecules.

In addition to having anti-inflammatory and tissue healing activities, the compositions provide a biochemical environment in accord with the law of mass action that can activate inactive genomic components and increase expression of one-third or more of the genome thereby potentially countering disease including hereditary conditions. This can counter a genetic imbalance and can therefore overwhelm disease-producing genes, even those produced by hereditary changes.

In addition the pharmacodynamic basis for the effects of genetic therapy non-chiral function in a self-perpetuating mode through the L tetrahedral 3D fit of L-amino acids and glycine non-covalent biochemical macromolecular binding to D polysaccharides such-as but not limited to the genetic system macromolecules DNA, RNA, ribosomal RNA and ribosomes and their respective polymerases furthered by the law of mass action mediated by progressive :therapy with synthetic therapeutic stem cell-like subject composition of L-amino acid and glycine. Thereby, in addition, these pharmacodynamic effects of genetic therapy function in a self-perpetuating mode through the biochemical law of mass action mediated by progressive therapy with synthetic therapeutic tissue and stem cell-like subject composition of L-amino acid and glycine, polar surface-active lipids and optional inclusion of extracellular matrix scaffold.

L-tetrahedral fit; Surfaces and Tetrahedral fit of each alpha amino acid. Surface magnification of molar ratio (protein) and reactive moieties and tetrahedral fit in protein synthesis and as therapeutic anti-inflammatory healing therapy,

The C2 through C6 twenty L amino acids and non chiral glycine including the 8 C3 propionic acid derivatives are analog to such C3 propionic acid derivative, and Ca4 3butyric acid derivative, anti-inflammatory medications and their reactive moieties. In contrast the routine anti-inflammatories listed in the PDR are benzene ring containing compounds from which many medications. and anti-inflammatory drugs are derived, lacking the L alpha amino acid and glycine 3D tetrahedron fit in protein synthesis, actually interfere with protein synthesis, (a non-tetrahedral 3D planar gliding action is present in anti-inflammatory medication).

The compositions can also be employed for metabolic diseases and conditions such as Type 1 with insulin deficiency wherein the molar ratio of the protein insulin may be incorporated into subject composition to stimulate the production of insulin as well as replacing suspected trace element deficiency such as but not limited to chromium or type 1 diabetes and the diabetic state where there is adequate insulin but with inadequate insulin receptor response which may be modified with high HLB therapy.

The therapeutic compositions may also be specifically applied to addiction by mimicking normal tissue metabolism and normal tissue including the L-amino acid glycine molar ratio of endorphin to metabolically stimulate and in fact coerce the body to produce this hormone. These same principles and therapeutic components have been applied in normalizing, as noted in a prior embodiment's dependency or withdrawal symptoms such as, but not limited to, the use of drugs in controlled substances, alcohol and/or drug and tobacco addiction in the medical patient or veterinary practice or experimental conditions such as the animal or tissue culture. Therefore these compositions form a clinical bridge beyond other advanced technologies that have not to date found a clinical application. Examples of suitable therapeutic uses include the treatment of Crohn's Disease, and in particular Pediatric Crohn's Disease (PCD), a chronic, relapsing, unremitting disease with grave, guarded prognosis for which conventional treatment includes high-risk immune suppressants such as corticosteroids at high doses. In many cases, particularly in pediatric cases, major surgical intervention is required within five (5) years of initiation of observation, with resection of up to several hundred grams of diseased organ tissue. Surgical intervention effectively arrests disease complications but has no effect on the clinical course of the disease. In fact, many patients require repeated surgical intervention. The use of these therapeutic stem cell-like subject compositions reduces or eliminates long-term corticosteroid use in these patients along with reducing side effects including but not limited to the interference and prevention of healing (so important in the management of Crohn's disease or Pediatric Crohn's disease) in these patients.

When these tissue normalizing principles and therapeutic subject compositions have been used in allergic asthmatic disease, therapeutic benefits have included: minimizing emergency use of corticosteroids, or possibly excluding the need for bronchodilator medication effect of sympathomimetic medication such as the beta sympathomimetic agonists. Further minimizing emergency use of sympathomimetic medications and their vicious cycle, of rhinitis medicamodosa or asthmatic bronchitis or potential bronchopulmonary equivalent asthmatic medicamentosa side effects seen with the past inhalation overuse of isoproterenol as the locked lung syndrome).

Additionally, transplantation or other surgery can be averted in congenital biliary atresia (CBA), a disease that is usually fatal if left untreated surgically. Even though CBA has an incidence of 300 cases occur annually in the U.S. this disease represents the most common rational for liver transplantation in the pediatric age group.

The co-use of subject composition with the many medications available and prescribed from the PDR extend synergistic pharmacodynamics of these subject compositions and may be integrated with the successful bio-efficacy of the therapeutic effects of the compositions, exemplified by:

(1) reinstitution of organ and tissue function regardless of organ and tissue involved and regardless of etiology, such as but not limited to trauma;

(2) diseases of inherited predisposition such as, but not limited to, lysosomal storage diseases and deficiency diseases such as but not limited to enzymatic deficiency including for example, lysosomal storage disease in addition to specific enzyme deficiency replacement, residual tissue and organ dysfunction due to encroachment of distended lysosomes may be further treated with these subject compositions. This includes HLB modulation with the added advantage of the polar surface active lipid surfactant high HLB packing parameter to synergize, facilitate and accelerate small amounts of enzyme that may be present. This is accomplished by increasing surface area, not only of the deficient enzyme, but also of its substrate to maximize the enzyme's metabolic activity. By these methods, the genetic profile and pattern predisposing to disease in treatment will be minimized and normal genetic function become more dominant. Include as exemplified here but not limited to even the recessive lysosomal storage diseases.

Diseases and the syndrome of diseases may be viewed here as being analog to an insoluble crystalline ‘thorn in the side’ of the patient's tissue and metabolic processes whether diseases such as obesity with insoluble fat particles, atherosclerosis with cholesterol crystals, cancer, genetic diseases lacking enzymes to fluidize and hydrophilize these lysosomal deposits, or other insoluble crystal like structures such as asbestos or silicosis. The liquid crystals provided in this discovery characteristic of the polar surface active lipids thereby reverses these disease mechanisms structures and functions whether by the highly hydrophilic polar surface active lipid surfactant and or by the initial in component dispersed at other fat bite highly lipophilic polar surface active lipid surfactant.

The added advantage offered by these surfactants is that by making these crystalline or crystalline like non-soluble metabolites randomly disbursed thereby changing entropy, energy is also provided at the same time equivalent to energy of metabolizing and fat such as palmitic acid or the combustion of paper with the release of energy to complete the metabolism of these disease causing crystalline structures.

Current medication in the public domain emphasizes the use of (as exemplified in cancer) of platinum and cis-platinum and other allied anti-cancer therapeutic agents. These agents were originally noted to be lethal to infectious microorganisms and this concept and was further translated to the therapy of cancer.

Singular and novel to the prior art is therapy for infectious disease or for cancer that is not dependent on its lethality to tissue and its associated disease but is dependent upon the principle that human tissue can be facilitated and synergized to assume the function and structure of replicating itself, thereby replacing the vicious cycle of disease. This averts major side effects difficult to accept that are associated with therapeutic lethality concept, thereby normalizing human tissue using compositions that mimic and are analog to human tissue not only in structure but also in function.

Such compositions can be used to treat neoplasms as in cancer or infectious diseases, in overcoming antibiotic sensitivity, or inactivation without damaging or killing human tissue. In the case of infectious disease, the same high HLB polar surface active lipid surfactant composition as in the anti-cancer therapeutic components as in No. 2 and are used to counter such microorganism invasive modalities as lipid A, LPS (lipo-polysaccharide as in toxic shock syndrome) that were formerly antibiotic resistant. A similar dual mechanism as with platinum however without major side effect concerns.

In inflammation and degenerative diseases without giving up imperative protein synthesis in healing associated with the existing anti-inflammatory drugs, synthetic therapeutic stem cell containing components Nos. 1, 2, 3, 4 and 5 can be used.

Congenital and genetic diseases can be treated using a composition of a “synthetic stem cell” containing therapeutic component Nos. 1, 2, 3, 4 and 5 without assuming life threatening entree through the portal vein and infectious microorganism carrier agents. For example, oral mucosal administration can be used, (thereby bypassing portal vein delivery) in these so targeted applications.

Trauma management can be performed with the local and systemic use of a synthetic stem cell composition containing component Nos. 1, 2 and 3, while greatly minimizing additional trauma and salvaging tissue by minimizing the requirement of debriedment. This provides a sutureless wound closure using progressive approximations with steri strips and inactivation of collagenase which has been activated by cortico-steroids, (which has become more commonly used in the management of chronic diseases).

These foregoing treatments combined as one therapeutic unit but administered as a single dosage or two to 4 times daily divided dosages may be given locally, systemically including intravenous administration and oral mucosal delivery system companion to this series of inventions.

Compositions of the subject invention can further comprise one or more compounds generally accepted as safe (GRAS) selected from the group consisting of aspartame perfluorocarbon resins, perfluorocarbon cured elastomers. [alpha]-Amylase enzyme preparation from Bacillus stearothermophilus, benzoic acid, bromelain, catalase (bovine liver), lactic acid, linoleic acid, potassium acid tartrate, propionic acid, stearic acid, tartaric acid, diacetyl tartaric acid esters of mono- and diglycerides, ammonium bicarbonate, ammonium carbonate, ammonium chloride, ammonium hydroxide, ammonium citrate, dibasic, ammonium phosphate, monobasic; ammonium phosphate, dibasic; bacterially-derived carbohydrase enzyme preparation; bacterially-derived protease enzyme preparation; bentonite; benzoyl peroxide; n-Butane and iso-butane; Calcium glycerophosphate; Calcium lactate; Calcium pantothenate; Calcium propionate; Calcium stearate; Carbon dioxide; Beta-carotene; Cellulase enzyme preparation derived from Trichoderma longibrachiatum; Clove and its derivatives; Cocoa butter substitute; Copper gluconate; Copper sulfate; L-Cysteine; L-Cysteine monohydrochloride; Dextrin; Diacetyl; Enzyme-modified fats; Ethyl alcohol; Ficin; Glucono delta-lactone; Corn gluten; Wheat gluten; Glyceryl monooleate; Glyceryl behenate; Glyceryl palmitostearate; Helium; Inositol; Insoluble glucose isomerase enzyme preparations; Isopropyl citrate; Animal lipase; Magnesium carbonate; Magnesium chloride; Magnesium hydroxide; Magnesium oxide; Magnesium phosphate; Magnesium stearate; Magnesium sulfate; Malt; Malt syrup (malt extract); Manganese chloride; Manganese citrate; Manganese gluconate; Manganese sulfate; Microparticulated protein product; Mono- and diglycerides; Monosodium phosphate derivatives of mono- and diglycerides; Niacin; Niacinamide; Nickel; Nitrogen; Nitrous oxide; Peptones; Pancreatin; Papain; Pectins; Pepsin; Potassium bicarbonate; Potassium carbonate; Potassium chloride; Potassium hydroxide; Potassium lactate; Propane; Pyridoxine hydrochloride; Rennet (animal-derived) and chymosin preparation (fermentation-derived); Riboflavin; Riboflavin-5′-phosphate (sodium); Sodium benzoate; Sodium carbonate; Sodium hydroxide; Sodium hypophosphite; Sodium lactate; Sodium metasilicate; Sodium propionate; Sodium sesquicarbonate; Sodium tartrate; Sodium potassium tartrate; Starter distillate; Stearyl citrate; Thiamine hydrochloride; Thiamine mononitrate; [alpha]-Tocopherols; Triacetin; Tributyrin; Triethyl citrate; Trypsin; Urease enzyme preparation from Lactobacillus fermentum; Vitamin A; Vitamin B12; Candelilla wax; Carnauba wax; Bakers yeast extract; Zein; Sulfamic acid; Clay (kaolin); Ferric oxide; Iron oxides; Japan wax; Tall oil; Alfalfa; Allspice; Almond, bitter (free from prussic acid); Ambrette; Angelica root; Angelica seed or stem; Angostura; Anise; Asafetida; Balm; Balsam of Peru; Basil; Bay leaves; Bay; Bergamot (bergamot orange); Bois de rose; Cacao; Camomile (chamomile); Capsicum; Caraway; Cardamom seed (cardamon); Carob bean; Carrot; Cascarilla bark; Cassia bark, Chinese; Cassia bark, Padang or Batavia; Cassia bark, Saigon; Celery seed; Cherry, wild, bark; Chervil; Chicory; Cinnamon bark, Ceylon; Cinnamon bark, Chinese; Cinnamon bark, Saigon; Cinnamon leaf, Ceylon; Cinnamon leaf, Chinese; Cinnamon leaf, Saigon; Citronella; Citrus peels; Clary (clary sage); Clove bud; Clove leaf; Clove stem; Clover; Coca ; Coffee; Cola nut; Coriander; Corn silk; Cumin (cummin); Curacao orange peel; Cusparia bark; Dandelion; Dandelion root; Dill; Dog grass (quackgrass, triticum); Elder flowers; Estragole ; Estragon (tarragon); Fennel, sweet; Fenugreek; Galanga (galangal); Garlic; Geranium; Geranium, East Indian Geranium, rose; Ginger; Glycyrrhiza; Glycyrrhizin, ammoniated; Grapefruit; Guava; Hickory bark; Horehound (hoarhound); Hops; Horsemint; Hyssop; Immortelle; Jasmine; Juniper (berries); Kola nut; Laurel berries; Laurel leaves; Lavender; Lavender, spike; Lavandin; Lemon; Lemon balm (see balm); Lemon grass; Lemon peel; Licorice; Lime; Linden flowers; Locust bean Lupulin; Mace; Malt (extract); Mandarin; Marjoram, sweet; Mate 1; Menthol; Menthyl acetate; Molasses (extract); Mustard; Naringin; Neroli, bigarade; Nutmeg; Onion; Orange, bitter, flowers; Orange, bitter, peel; Orange leaf; Orange, sweet; Orange, sweet, flowers; Orange, sweet, peel; Origanum; Palmarosa; Paprika; Parsley; Pepper, black; Pepper, white; Peppermint Peruvian balsam; Petitgrain; Petitgrain lemon; Petitgrain mandarin or tangerine; Pimenta; Pimenta leaf; Pipsissewa leaves; Pomegranate; Prickly ash bark; Rose absolute; Rosa; Rose; Rose buds; Rose flowers; Rose fruit (hips); Rose geranium; Rose leaves; Rosemary; Rue; Saffron; Sage; St. John's bread; Savory, summer; Savory, winter; Schinus molle; Sloe berries; Spearmint; Spike lavender; Tamarind; Tangerine; Tannic acid; Tarragon; Tea; Thyme; Triticum; Tuberose; Turmeric; Vanilla; Violet flowers; Violet leaves; Violet leaves absolute; Wild cherry bark; Ylang-ylang; and; Zedoary bark, or any combination of said compounds. Any combinations of the compounds GRAS can be used in formulating compositions of the subject invention. In some embodiments, the composition further comprises a flavorant that can be a fruit juice, such as tomato juice.

The following sections of Title 21 of the Code of Federal Regulations are hereby incorporated by reference in their entireties (with respect to materials generally recognized as safe (GRAS)): §§ 5, 25, 170, 172, 173, 177, 182, 184, 186, 570, and 582.

This tissue healing tissue regeneration therapy can be used in conjunction with many other therapies that have dramatic therapeutic effects and high incidence of side effects that has heretofore minimized their popularity and value. Administration of this synthetic stem cell-like subject composition to these patients can protect them from these side effects and the side effects can be greatly minimized. The same therapeutic and prophylactic concept can be used in the unfortunate possibility of a bioterrorism attack, (mediated by nuclear material, microorganisms, chemical agents) or in speeding tissue healing and tissue regeneration of soldiers on the battlefield.

### EXAMPLES

The following examples are used to illustrate preferred embodiments of the invention and are not meant to limit the scope of the invention in any way.

In medicine, the average dosages are determined from a bell-curve. For example, most of the patients might respond to dosages as given. However, the beginning of the bell curve response might be 10% to 50% of these dosages and the end of the bell curve might be 125% to 200% of these dosages. Further results may be augmented by addition of component No. 4 and No. 5 to compositions comprising components Nos. 1-3. In addition, this provision applies to all examples included by reference of Patent Ser. Nos. 60/149,338, 09/639,859, 10/752,298, and 10/765,664.

### EXAMPLE 1

### Example of Multi-Center Study

Within three to six weeks of initiation of this treatment using the compositions of the invention approaching 95% of Crohn's disease (CD) and pediatric Crohn's disease (PCD) patients are being studied in double-blind, placebo-controlled multi-center including 35 radiographically tagged inflammatory neutrophile permeability studies as well as the open study are able to discontinue the immune suppression therapy.

Remarkably, the discontinuance is not associated with relapse for a period of at least six months. More than 95% are maintained in the disease-free state for as long as one year. Growth arrest and puberty suppression are overcome within six weeks in more than 20 patients with a predictable efficacy of over 95% in 150 patients (⅓ of 450 being studied, (PCD cases treated exceed 200 patients). Controlled in-vitro tissue culture studies of biopsied Crohn's tissue evidences significant (approaching more than 95%) reduction in inflammatory mediator chemokines relative to controls after 24 hours.

The compositions also avert the need for surgery to address another Crohn's Disease complication (intestinal fistulae) in more than 95% of the cases, and in those cases that require surgery in more than 95% (reduction surgical mortality with use of subject composition).

Also, in medicine the average dosages are determined from a bell-curve. For example, most of the patients might respond to dosages as given. However, the beginning of the bell curve response might be 10% to 50% of these dosages and the end of the bell curve might be 125% to 200% of these dosages.

In medicine, the average dosages are determined from a bell-curve. For example, most of the patients might respond to dosages as given in example 2 (infant and child). However, the beginning of the bell curve response might be 10% to 50% of these dosages and the end of the bell curve might be 125% to 200% of these dosages.

### Example 2

Congenital biliary atresia (CBA) is a rare fatal (prior to treatment with this invention) disease without liver transplant, with a U.S. incidence of only 300 cases per year (approximately 1 per 1 million of U.S. population) with symptoms occurring at onset of infancy that include: poor appetite, poor food intake, extreme jaundice, (21 mgm percent bilirubin, with biliary obstruction further confirmed by an abnormal dye excretion study. stools were gray, acholic, lacking normal stool bilirubin color), lassitude, weight loss, failure to thrive, abnormal liver function tests, abnormal liver ultrasound, and abnormal biopsy.

Therapy with the compositions of the invention in a CBA patient using components No. 1 and No. 2 re-established organ function, stimulated tissue healing and tissue protein synthesis concurrent with significant anti-inflammatory activity, clearance of all abnormal liver function, and averted the required for a liver transplant

Components No. 1 and No. 2 were used and the composition comprised of 20 to 30 grams L-amino acids and glycine in the molar ratio of breast milk suspended in 4 oz. of water. Four to 6 doses administered daily were sufficient to normalize, over a period of 3 months all abnormal liver function studies, abnormal liver ultrasound, abnormal biopsy, as well as reversing symptoms of jaundice, poor appetite, poor food intake, lassitude, weight loss, and failure to thrive, off liver transplant list. The patient was sent home with happy disposition, not crying normal stools, sleeping well and easily burped.

Component No. 3 can also be added in the dosage of 0.5 to 2 grams per day, and optional components No. 4 (¼ to ½ of the dosages as administered in Example 3b) and component No. 5 can (¼ to ½ the dosage as administered in Example 3b) can also be added to compositions of the subject invention.

### Example 3

### Crohn's Disease, (CD) Case Report, Part (a)

A 71 year old female patient with more than 3 decades of Crohn's disease whose symptoms included diarrhea, constipation, severe bouts of abdominal pain and fever, generalized aching, extreme fatigue, nausea, food and dairy intolerance, increased sedimentation rate, recently had a flare up of the Crohn's disease. Response from 4 mgm of corticosteroid, once daily was unsatisfactory. Corticosteroid dosing was then increased to 4 times daily for acute flare ups.

The patient received a composition comprising 5 to 25 grams of L-amino acids and glycine, lecithin (phospholipid-PC), and extracellular matrix components comprising collagen, proteoglycan aggregate complex of cartilage and chondrotin sulfate (shark cartilage 740 mg. per capsule, 4 capsules twice daily). Symptoms of severe abdominal pain and diarrhea, and the flare-up were cleared within 24 hours. The improvement continued over the next few weeks, and the patient responded to the least amount of corticosteroids (alternating daily dosages of a half a tablet (2 mg) with a full tablet (4 mg) required to prevent flare-ups in the past several decades of management.

This reduction in steroid dosage has also reduced severe unsightly bruising and poor healing of lacerations and associated intolerance of sutures. Her lacerations have been most successfully healed with non-suture steri strips.

The second therapeutic component comprises 2.1 grams of omega 3 seed oil, (flax oil, sunflower oil, sesame seed oil 1.7 grams of omega 6 oil, and 1 gram omega 9 oil (Flora brand) (with the following well tolerated preferred recent substitution of omega 3 fish oil and seed oil for just few weeks: 2 capsules 1-2 times daily, Thera Tears, serving size 2 softgels per serving, containing per 2 capsule Vitamin E (as d-alpha tocopherol concentrate) 100 IU (anti-rancidity antioxidant), Organic Flaxseed Oil 500 mg, EPA (Eicosapentaenoic Acid) (from Fish Oil) 225 mg, and DHA (Docosahexaenoic Acid) (from Fish Oil) 50 mg. The anti-rancidity antioxidant vitamin E present in this capsule prevents the development of catabolic products that are counter to the components of this therapeutic innovation accounting for the tolerance of this fish oil product.

This patient is one of the unusual patients intolerant to fish oil. Patients with ileitis have a deficiency of pancreatic lipase and enteric coated fish oil capsules may be more helpful in overcoming this intolerance. This anti-inflammatory immune modulatory pharmacologic activity is furthered by the addition of vitamin A (5,000 units), 250 ml of vitamin C, 400 ml vitamin E (d alpha tocopherol), selenium (20 mg) and Zinc (15 mg).

It should be noted here that significant progress has been made here and in these foregoing embodiments in masking a major problematic taste of the amino acid component which formerly, in the prior art, brought about the requirement of gastric tube administration and associated hospitalization.

Encapsulation of the medication would eliminate use of the gastric tube by by-passing the problematic taste of the amino acid component. However, for the pediatric or adult patient who can not take capsules, a vegetable flavored juice such as, but not limited to, tomato juice or V8 could be used as a flavored vehicle. One heaping teaspoon (approximately 5 grams) to 5 ounces of juice, was found by a taste panel to thoroughly mask the most objectionable taste of the first component, the amino acid product. This amino acid component includes, but is not limited to, Neocate for Infant use. This Crohn's patient was included in our taste panel in our attempt to improve the palatability of the objectionable amino acid component of subject composition.

### Example 3

### Part (b) Crohn's Disease

Further response to addition of therapeutic components No. 4 and No. 5 (All 5 component therapeutic composition response).

Further progress report and addition of components No. 4 and No. 5 to this patient care added even further to significantly improve her clinical course. The addition of components No. 4 and No. 5 have provided for normalization of enzyme composition secretion of the tissue and the normalization of the micro-organism flora with associated normalization of function of this gastrointestinal Crohn's diseased tissue has made possible for this patient for the first time to further reduce from one tablet of the corticosteroid that this three component therapy has permitted to use ½ tablet of corticosteroid (triamcinalone generic) for the first time in three decades without usual further steroid withdrawal symptoms of arthralgia common in steroid withdrawal as noted repeatedly in this patient in the past unsuccessful attempts of steroid reduction.

The side effects this patient has sustained from long-term corticosteroids has been worsening of osteoporosis documented by two successive bone scans two years apart, recurrent bruising and failure to heal including two threats of the need for skin graft which this subject composition stem cell-like treatment has prevented. Bruising and healing time of skin trauma as well as GI flare ups of diarrhea greatly improved.

### Example 3

### Part (c) Crohn's Disease

Further response to inclusion of additional beneficial bacteria to Component No. 5.

The addition of lactic acid bacteria (VSL #3Tm, commercially available from VSL Pharmaceuticals of Galthersburg, Md.) to component No. 5 further improved the clinical course for this patient (Example 3, part (c)). Packets of approximately 450 billion freeze dried yogurt bacteria were added to component No. 5 and given 2-3 times per day. This allowed the patient to further reduce corticosteroid use to 2 mg (½ of a 4 mg tablet) three times per week, slightly less than 1 mg per day on average without experiencing steroid withdrawal symptoms.

### Example 4

### Countering Wound Healing Impairment with Steroids

The prior embodiments documented the reversal of the need for skin graft in wound treatment of a Crohn's patient (exemplified by adding deficient vitamin A locally to anabolic counter collagenase stimulated by long-term corticosteroids) along with wound healing when zinc, in the form of zinc oxide, was added to composition No. 5.

### Example 5

### Orthopedic and Anti-Arthritic Subject Composition Therapy—Case Report

A female patient age 48 has been treated for acute degenerative arthritis right hip confirmed by x-ray findings. Acute onset, May of 02 associated with progressive pain limping and requirement of support with a cane temporarily relieved by anti-inflammatory drug Vioxx with x-ray findings of severe inflammatory degenerative arthritis associated with absence of joint space of right hip joint and clinical regression right hip. Joint prosthetic replacement even though only age 48 was recommended by rheumatologist and orthopod. Patient refused surgical care and responded with use of extracellular matrix:

ECM: Glucosamine 750 mg. daily, Shark cartilage 450 mg., Cartilage 50 mg, gelatin, a denatured collagen, porcine origin, 1 to 2 tablespoons in fruit juice.

The addition of an anti-inflammatory immune modulator (omega 3 flax-seed oil, 1000 mg) provided a progressive response with reappearance of the hip joint space on x-ray (severe inflammatory changes had interfered with visualization of any joint space). Since the patient is now pain-free and no longer requires a cane supportive of walking, however still had a mild limp, the completion of the synthetic stem cell first and second component chiral L amino acid and non chiral glycine in the molar ratio of human tissue supportive of the stem cell, along with polar surface active lipid as phospholipid lecithin was suggested in the form of Neocate progressing from 5 grams daily to 15 grams daily to three times daily. It is expected that this additional therapy should significantly add to the therapeutic response progression.

Anti-inflammatory, immune modulatory bio-efficacy, biosafety and pharmacologic activity is present with all four components of synthetic stem cell therapeutic subject composition.

### Example 6

### Inactivation of Cat Dander Allergen of Cat, to Lessen Respiratory Allergy Symptomatology after Cat Exposure (With Therapeutic Component No. 2)

High HLB liquid crystalline phase semi-conductor bio-computer used here and its biophysical hydrophilic micellar counterpart with its anti-allergenic subject composition therapeutic embodiments, in vitro basophilic de-granulation measure by histamine release comparing efficacy of treated cat dander in preventing histamine release with untreated cat dander when exposed to serum from cat allergic patients.

### Example 7

The use of high HLB surfactant in cancer may be used alone or as an optional component of component No. 2.

Comparative studies of inactivation of in-vitro cancer using tissue culture techniques with high HLB surfactant, Tween 80 are illustrated in Table 2.

Results of Treating T47D breast cancer tissue cells (Normalized):

TABLE 2
MTS (Breast cancer Mitochondrial activity Assay)

| | | | % |
|---|---|---|---|
| | | | normalization |
| | | | of Breast |
| Culture Time | 24 h | 48 h | cancer cells |
| Control | 1.0 | 1.0 | 0% |
| **Tween 80 (0.125%) | 0.48 | 0.24 | 76% |
| PC (0.125%) | 0.92 | 0.98 | |
| Tween 80 + PC | 0.61 | 0.42 | 58% |
| (0.125% + 0.12596) | | | |
| GS-1 (grape seed | 0.29 | 0.17 | 83% |
| Extract, 0.5 mg 1-ml | | | |
| Tween 80 + GS-1 | 0.27 | 0.17 | 83% |
| PC + GS-1 | 0.34 | 0.17 | 83% |

Breast cancer (Comparative histopathologic studies before and after treatment with high HLB Tween 80 surfactant).
**Histopathologic studies correlate with a more than 50% reduction of cancer cells seen after 48 hours of treatment with 0.125% Tween 80.

### Summary of Cell Inhibition Assays

Development of metastatic cancer involves several steps, usually separated in to initiating and promotional steps. Initiation involves somatic mutation leading to altered expression of genes controlling DNA synthesis and cell replication. Promotion involves stimulation of the mutated cell to continued division. Subsequent mutations in these altered cells lead to more aggressive replication and invasion of neighboring tissues. In many tumors, the tumor cells are cycling while their neighbors are in the GO phase of the cell cycle. Substances which interfere with mutagenesis or with cell division could prove to be anti-carcinogenic.

Several extracts of these for their abilities to inhibit the metabolism of cells isolated from breast and cervical cancer tissue have been examined in regard to the anti-cancer effects of extracts which have proven capable of significantly inhibiting the metabolism of these cancer tumor cells. In addition to and equal to the effects of high HLB surfactants for comparative testing. These comparative studies were performed and results are reported in the above table. Metabolism was comparatively measured with controls and other extracts as to the reduction of mitochondrial activity, (MTS). This compound is a substrate for the mitochondrial enzymes—and is reduced to a blue formazan product.

Activity of the extracts was tested with the MCF-7 and T47-D breast cancer cell lines and against the CaSki and SiHa cervical cell lines. During the later experiments, the CRL 7367 and CRL 7368 cell lines became available and were included in subsequent trials. CRL 7368 is a line established from transformed fibroblasts isolated from a breast cancer. CRL 7367 was established from apparently healthy skin fibroblasts taken from the same donor.

### Methods

In the first experiments—The extracted therapeutic test agents were derived from specified tissue. Water extracts of the crushed tissue, were also prepared. For the later experiments, specific varieties of extracts were prepared and examined: Cells were obtained from American Type Culture Collection (ATCC, Washington, D.C.) and were cultured as recommended by ATCC. Experiments were performed in 96 well microtiter plates. Each well contained 1.0×104 cells suspended in a total volume of 200 ml. Test wells contained the designated amount of extract in cell culture medium. Control wells contained the same amount of extract solvent in medium. Plates were incubated in an atmosphere of 5% C02 for 24 or 48 hours with extract or solvent. At this time, -pl -(MTS) were added to each well and incubation was continued for an additional 4 hours after which time the optical density (OD) at nm of each well was recorded. In each experiment, each sample was assayed in triplicate and the mean optical density (OD) for the three wells containing the same culture was calculated.

### Results

Data are presented as suppression ratios. The suppression ratio defined here as the mean optical density (OD) for the wells containing extract divided by the mean optical density (OD) for the control wells. A value of this ratio of 1.0 indicates that the extract had no effect on metabolism of the cells being tested. A value of less than 1.0 indicates inhibition of cell metabolism by the extract or the high HLB surfactant Tween 80.

The purpose of the work was to determine which therapeutic agent extracts (further separated by chromatography) explored through this methodology for anti-cancer activity. The data presented here represents semi-quantitative anti-cancer screening tests. Extracts with ratios in the range 0.51-0.74 were considered moderately active and therefore appropriate for considered use along with preventive anti-cancer treatment and active anti-cancer treatment. In co-use with anti-cancer treatment and radiation treatment, may lessen the dose and the side effects of these current therapeutic agents. Extracts with metabolism suppression ratios less than or equal to 0.5 represented a suppression of metabolism of at least 50% or greater than that of the controls and were considered definitely active potential anti-cancer agents, as was the case of High HLB Tween 80 with ethylene maturation apoptosis promoting factor with a 76% suppression of mitochondrial metabolism of cancer cells.

In the first experiments, tissue extract prepared in the laboratory and water extract from tissue commercially obtained were, comparatively examined. These results were presented in Table 1. The data indicated that comparatively both therapeutic agents derived from tissue extracts freshly prepared in this laboratory and water extract inhibit can cell metabolism at the higher concentrations tested. There is a clear dose effect indicating that therapeutic agents derived from tissue extracts prepared in our laboratory at concentrations lower than 0.004 and commercially available water extract concentrations lower than 0.02 do not inhibit metabolism. Data for alcohol extracts were also presented. The extracts had minimal effects on metabolism after three days of treatment, but after five days of treatment the ethanol extract had inhibited metabolism of both breast cancer cell lines by over 60%. Some extracts suppressed the MCF-7 cell One, but had minimal effect on the T47-T cell line or on the cervical cancer cell lines even when the extract anti-cancer treatment agent composed 4% of the total culture volume. Of all the extracts examined the tissue extract anti-cancer treatment agent obtained with 70% acetone/30% water was the most active.

Acetone is an agent used in separating phospholipid surfactants, e.g. phosphatidylcholine (PC), phosphatidyl serine (PS), phosphatidylinositol (PI), and phosphatidylethanolamine (PE) acetone insoluble representing 58% of surfactants present in soy lecithin. 70% acetone and 30% water used here as a tissue extracting agent is most probably a hydrophilic surfactant.

### Example 8

### Case Report—Therapeutic Composition to Counter Withdrawal Symptoms and Side Effects of Medications and Drugs and Drug Addiction and Dependency

The Therapeutic Results and Rationale for inclusion of Components No. 4 and No. 5: This detailed therapeutic replication of normal human tissue (and therefore complete reversal of disease tissue) and by including the products of therapeutic component Nos. 4 and 5, (added to component Nos. 1, 2, and 3 past month and added past two months to care of prior patient), normalization of enzyme composition secretion of the tissue and the normalization of the micro-organism flora with associated normalization of function of this gastrointestinal Crohn's diseased tissue has made possible for this patient for the first time (and not reported or taught in that art) to further reduce from one tablet of the corticosteroid that this three component therapy has permitted to use ½ tablet instead of corticosteroid (triamcinalone, common generic) for the first time in three decades without usual further steroid withdrawal symptoms of arthralgia common in steroid withdrawal as noted repeatedly in this patient in the past unsuccessful attempts of steroid reduction.

The side effects this patient has sustained from long-term corticosteroids has been worsening of osteoporosis documented by two successive bone scans to years apart, recurrent bruising and failure to heal including two threats of the need for skin graft which this subject composition stem cell-like treatment has prevented. Bruising and healing time of skin trauma as well as GI flare ups of diarrhea greatly improved.

These therapeutic compositions may also be specifically applied to addiction by mimicking normal tissue metabolism and normal tissue including the L-amino acid glycine molar ratio of endorphin to metabolically stimulate and in fact coerce, by the law of mass action, the proteins assemblage system of the body to produce this hormone. Since one mole of tyrosine, two moles of glycine and one mole of phenylalanine seem to be essential for the narcotic effects of beta endorphin and the met and leu-enkephalins, this anti-addiction effect then would be compared to the complete L-amino acid glycine molar ratio of beta endorphin. This molar ratio of beta endorphin also includes one mole of methionine, two moles of threonine, two moles of serine, four moles of lysine, two additional moles of phenylalanine, one mole of glutamine, one mold of proline, one mole of valine, one mole of leucine, two moles of asparagine, two moles of alanine, two moles of isoleucine, one mole of tyrosine, one mole of glycine, and one mole of glutamic acid.

The same principles and therapeutic components have been applied in normalizing, as noted in prior embodiments, dependency or withdrawal symptoms such as, but not limited to, the use of drugs in controlled substances, alcohol and/or drug and tobacco addiction in the medical patient or veterinary practice or experimental conditions such as the animal or tissue culture.

Therefore, these therapeutic compositions form a clinical bridge beyond other advanced technologies that have not to date found a clinical application with exemplary bio-safety.

A variety of modifications to the embodiments described will be apparent to those skilled in the art from the disclosure provided herein. Thus, the invention may be embodied in other specific forms without departing from the spirit or essential attributes thereof and, accordingly, reference should be made to the appended claims, rather than to the foregoing specification, as indicating the scope of the invention.

1. An anabolic medicament for treating a damaged tissue, the medicament comprising:
a first component comprising a plurality of L amino acids;
a second component comprising at least one extracellular matrix compound,
a third component comprising at least one polar surface active lipid;
a fourth component comprising at least one vitamin, mineral or trace element; and
a fifth component comprising a probiotic;
the fourth and fifth components synergistically interacting with at least one of the first through third components to promote repair of damaged tissue.

2. The medicament of claim 1, wherein the probiotic comprises a plurality of beneficial microorganisms.

3. The medicament of claim 1, wherein the probiotic comprises an ingredient that promotes the growth of beneficial microorganisms.

4. The medicament of claim 1, wherein the probiotic comprises lactic acid bacteria.

5. The medicament of claim 1, wherein the probiotic comprises an enzyme.

6. The medicament of claim 5, wherein the enzyme is a human digestive enzyme.

7. The medicament of claim 6, wherein the human digestive enzyme is a pancreatic enzyme.

8. The medicament of claim 1, wherein the plurality of L amino acids are present at a molar ratio which is characteristic of human breast milk.

9. The medicament of claim 1, wherein the polar surface active lipid is phosphatidylcholine.

10. A method of treating a patient having a damaged tissue, the method comprising the steps of:
administering to the patient a medicament comprising:
a first component comprising a plurality of L amino acids;
a second component comprising at least one extracellular matrix compound,
a third component comprising at least one polar surface active lipid;
a fourth component comprising at least one vitamin, mineral or trace element; and
a fifth component comprising a probiotic;
allowing the fourth and fifth components to synergistically interact with at least one of the first through third components to promote repair of damaged tissue.

11. The method of claim 10, wherein the fifth component further comprises a human digestive enzyme.

12. The method of claim 11, wherein the polar surface active lipid comprises a high hydrophile-lipophile balance surfactant that synergizes enzymatic activity associated with the fifth component.

13. The method of claim 12, wherein the hydrophile-lipophile balance is greater than about 13.

14. The method of claim 10, wherein the first through fifth components interact with one another after administration to mimic the effects of stem cell therapy.


SEQUENCE LISTING (1 sequence)

SEQ ID NO: 1 - protein, 152 aa, Homo sapiens
GLSDGEWQLVLNVWGKVEADIPGHGQEVLIRLFKGHPETLEKFDKFKHLKSEDEMKASEDKKHGATVLTALGGILKKKGHHEAETKPLAQSHATKHKIPVKYLEFISECIIQVLQSKHPGDFGADAQGAMNKALELFRKDMASNYKELGFQG